(19) United States
(12) Patent Application Publication
(10) Pub. No.: US 2007/0254286 A1
(43) Pub. Date: Nov. 1, 2007

(54) Molecular Markers that predict breast cancer development

(75) Inventor: Indira Poola, North Potomac, MD (US)

Correspondence Address: DUANE MORRIS, LLP;IP DEPARTMENT, 30 SOUTH 17TH STREET, PHILADELPHIA, PA, 19103-4196, US

(73) Assignee: Silbiotech, North Potomac (US)

(21) Appl. No.: 11/413,911

(22) Filed: Apr. 28, 2006

(51) Int. Cl. C12Q 1/68; G01N 33/574

(52) U.S. Cl. 435/6; 435/7.23

(57) ABSTRACT

A number of selected genes/gene products; Application of selected genes/gene products at mRNA or protein levels either singly or in combination; Application of selected genes/gene products at mRNA levels by any of the methods such as: Northern blotting, or reverse transcription and conventional PCR, or reverse transcription and quantitative real-time PCR or gene expression micro-arrays; Application of selected genes/gene products at protein levels by either Western Blotting, or immunohistochemistry, or ELISA or functional assays or gel electrophoretic separation followed by spectroscopic identification (proteamics); Application of selected genes/gene products at peptide levels derived from proteins and spectroscopic methods of identification; Detection of a hyperproliferative condition, a precancerous condition, a predisposition to develop hyperproliferative condition or cancer by applying any one of the selected genes either singly or in combination in breast tissue, breast fluid, breast cells, blood or any other tissues or cells of a mammal; Application of selected genes either singly or in combination with others for designing molecular therapeutic drugs to treat a hyperproliferative condition, a precancerous condition, or predisposition to develop hyperproliferative condition or cancer of the breast or any other tissue of a mammal; Application of selected genes either singly or in combination with others for following up of a therapeutic treatment to a hyperproliferative condition, or precancerous condition, or predisposition to a hyperproliferative condition, or cancer, of the breast or any other tissue of a mammal; Application of selected genes either singly or in combination with others for screening of therapeutic drugs to a hyperproliferative condition, or precancerous condition, or predisposition to a hyperproliferative condition, or cancer, of the breast or any other tissue of a mammal; and Application of selected genes either singly or in combination with others for designing vaccines to prevent a hyperproliferative condition, or precancerous condition, or predisposition to a hyperproliferative condition, or cancer, of the breast or any other tissue of a mammal.

### FIELD OF THE INVENTION

This invention pertains to isolated genes and the use of these gene products, both mRNA and protein levels, either singly or in combination, for determining a hyperproliferative condition, or a predisposition to a hyperproliferative condition, such as breast cancer, for prognosticating response to a therapeutic treatment of breast cancer or determining predisposition to develop breast cancer, treatment design, follow up on response, for screening candidate therapeutic treatments for a predisposed condition and for designing vaccines to prevent development of a hyperproliferative condition.

### BACKGROUND OF THE INVENTION

Invasive breast cancer (IBC) is the most diagnosed cancer and the second leading cause of cancer deaths for women in the United States. In the year 2005, about 217,440 women were diagnosed with IBC and an additional 59,000 with Ductal Carcinoma In Situ (DCIS) and the death rate was about 40,000 (Jamal, A. et al, CA Cancer J. Clin. 56: 10-30 (2005)). Although the mortality rate has slightly declined in recent years, primarily due to increased awareness and early detection, it still remains very high, mainly due to our limited success in curing the cancer completely once it has developed. It is rational to propose that death rate could be further reduced if the cancer is detected and treated at the precancerous stage. For this reason, there is a great deal of interest in understanding the biology of precancerous lesions that have the potential to develop into IBC, so that methods to detect at the precancerous stage and effective preventive treatment strategies can be developed.

Several lines of evidence indicated that development of invasive breast cancer is a multi-step process. Based on animal experiments and epidemiological evidence from humans, it has been proposed that stem cells in terminal duct lobular units (TDLU) undergo proliferation to hyperplasia without atypia, to atypical hyperplasia, to carcinoma in situ and IBC (FIG. 1) (Allred, D. C. et al. Endocrine-Related Cancer. 8, 47-61 (2001), Krishnamurthy, et al Advances in Anatomic Pathology. 9: 185-197 (2002)). Several retrospective and prospective studies of breast biopsies and mastectomy specimens have provided an indirect evidence that atypia and carcinoma in situ occur more often in the cancerous breasts than non-cancerous breasts (Ryan, J. A. et al, Cancer J. Surge. 5: 2-8 (1962), Karpus C. M. et al Ann. Surg. 162: 1-8 (1995)) and relative increased risk of developing carcinoma in a woman with usual ductal hyperplasia with out atypia (UDH) was 2.0. If the hyperplasia was associated with atypia the risk increased to 5.0 (Black, M. M. et al Cancer. 29: 338-43 (1972), Dupont, W. D. et al N. Engl. J. Med. 312: 146-51 (1985), Dupont, W. D. et al Cancer, 71:1258-65 (1993), London, S. J. et al JAMA, 267: 941-4 91992)). The incidence of atypical lesions is multi-focal and their increased incidence in cancerous breasts rather than non-cancerous breasts has suggested that they are precancerous lesions (Foote, F. W. et al Annals of Surgery, 121: 197-222 (1945), Wellings, S. R. et al J. Natl. Cancer Inst. 55: 231-243 (1975)). It is now well established that a majority of breast cancers arise in the milk ducts and, usual ductal hyperplasias (UDH) atypical ductal hyperplasias (ADH) are the most prevalent precancerous lesions that have the significant increased potential to developing into IBC.

Detection of precancerous/atypical cells before the formation of malignant breast tumors has become a reality since the development of methods such as ductal lavage collection and ductal endoscopy procedures. In recent years, atypical lesions are being diagnosed either endoscopically or cytologically by examination of cells from ducts, in addition to conventional histological examination of benign/atypical breast lesions. However, detection of atypical cells does not necessarily signal a precancerous stage since epidemiological evidences have shown that not all atypical lesions have the potential to become IBC. Some revert to an even less advanced phenotype. Since all atypical lesions look alike cytologically/histologically, it is not possible to predict which sub-class has the potential to progress to IBC based on the morphological appearance alone. Several epidemiological evidences have also demonstrated that ADH is not an obligatory step in developing IBC, and cancer could arise from morphologically non-atypical/normal appearing cells. Absence of atypical cells also does not exclude the presence of precancerous cells since not everyone who develops IBC had a history of ADH. Thus, currently, there are no means of precisely identifying a ‘True Precancerous’ stage. If molecular markers that are present in the ‘True Precancerous’ cells are known, it will significantly contribute in detecting the presence of these cells instead of relying solely on morphology, which seems to be very subjective. However, no such molecular markers that could predict breast cancer development were known.

In addition to detection of precancerous cells, molecular targets need to be identified for designing prophylactic drugs to prevent precancerous cells from becoming IBC. Currently, tamoxifen is the only prophylactic drug that has been approved for high risk women with ADH lesions or more recently atypical cells by ductal lavage are recommended to receive anti-estrogen, tamoxifen, therapy (Tan-Chiu, E. et al. Natl. Cancer. Inst. 95, 302-307, 2003). A STAR trial that treats with tamoxifen and raloxifen for preventing cancer in women with prior ADH is also in progress. Tamoxifen is also recommended for women with other type of benign conditions such as fibroadenoma, fibrocystic change, fibrosis and hyperplasia with out knowing whether they will develop cancer or not. The above preventive therapy is also recommended for post-menopausal women with modest risk as measured by Gail model (Veronesi, U et al. J. Natl. Cancer Inst. 95, 160-165, 2003, and IBIS Investigators Lancet, 360, 817-824, 2002)) Although tamoxifen was shown to reduce the incidence of breast cancer in women with prior ADH and non-ADH benign lesions, it is given non-discriminately without knowing whether these patients have ‘True Precancerous Lesions’ that develop into invasive cancers. As a result a large number of women are subjected to unwanted side effects (Fisher, B et al J. National Cancer Institute, 90, 1371-1388 (1998)) such as pulmonary embolism, deep vein thrombosis, stroke and endometrial cancers to tamoxifen. Unnecessary treatment with tamoxifen and the associated side effects could be avoided in a large percentage of women with benign lesions if there is a way to determine which patients will develop invasive cancer. The work proposed here will provide that information.

Although this drug has been shown to reduce the incidence of IBC, some women have had developed IBC even after receiving this drug and it is undesirable to younger women since it induces premature onset of menopause. For these reasons, there is a great deal of interest in identifying new molecular targets in precancerous breast lesions for designing of novel prophylactic drugs.

The present invention provides such markers that can predict development of breast cancer. In addition, the present invention also provides molecular targets to design prophylactic drugs to treat precancerous lesions and prevent IBC development. The present invention also provides molecular targets for designing vaccines ti prevent cancer development in patients with prior ADH or UDH lesions or a predisposition to develop cancer. This and other objectives and advantages of the invention, as well as additional inventive features, will be apparent from the description of the invention provided herein.

### BRIEF SUMMARY OF THE INVENTION

The present invention provides a selected number of genes/gene products mRNAs or proteins for detecting a predisposed tissue/cell for a condition, such as breast cancer in a mammal.

Further provided by the present invention is a selected number of genes/gene products mRNAs or proteins for prognosticating response of a mammal to a therapeutic treatment of hyperprolific condition, such as breast cancer, or a predisposition to a hyperprolific condition in a mammal.

Still further provided by the present invention is genes/gene products mRNAs or proteins as targets for designing therapeutic drugs for a hyperprolific condition such as breast cancer or a predisposition to a hyperprolific condition in a mammal.

Further provided by the present invention is a method of screening candidate therapeutic treatments for a hyperprolific condition, such as breast cancer or predisposition to a hyperprolific condition in a mammal using the gene products of the current invention. The method comprises comparing the expression profile of the isolated gene products before treatment with the expression profile of the tissue sample after treatment.

A method of determining a hyperproliferative condition or a predisposition to a hyperproliferative condition in a mammal is further provided by the present invention. The invention comprises comparing the expression profiles of the isolated genes or gene products in a hyperprolific condition such as a breast disease/cancer, with the normal tissue. The difference in the expression profiles of the mammal in comparison to the expression profiles of the control normal standard is indicative of a hyperproliferative condition or a predisposition to a hyperproliferative condition in a mammal.

Still further provided by the present invention is genes/gene products mRNAs or proteins as targets for designing vaccines to prevent a hyperprolific condition such as breast in a mammal.

### BRIEF DESCRIPTION OF THE DRAWINGS

FIG. 1 represents a schematic representation of currently accepted model of breast cancer progression paradigm (stages of breast cancer progression). Normal duct progressing to hyperplasia without atypia, hyperplasia to atypia, atypia to ductal carcinoma in situ (DCIS) and Invasive breast cancer (IBC).

FIG. 2 represents Principal Component Analysis (PCA) of ADHC (ADH from patients with a history of cancer) and ADH (control premalignant tissues from patients who had no previous history of cancer and did not develop cancer in 5 or more years after diagnosis) gene expression patterns. All genes called Present in at least 50% of the arrays (12330) were used in the analysis. The projection on the three principal components representing highest variance (62%) is shown.

FIG. 3 represents a table of names of genes with full descriptions and their abbreviations of the present invention that are to be used for detecting, prognosticating, vaccine designing, drug designing and screening drug targets for a hyperproliferative condition such as breast cancer or a predisposition to breast cancer in a mammal.

FIG. 4 represents Hirarchial clustering of unique genes having top 35 and bottom 35 ADHC/ADH ratios and 5 interesting genes (BCL.2A1, BIRC1, TACC3, CEACAM5, and TYMS) by average linkage and centered correlation. Logarithmic signal values were mean centered for each gene. Each point in space represents one array. The cluster is color coded using red for up-regulation and green for down regulation and black for medium expression.

FIG. 5 represents Gene Ontologies. Over-representation analysis of gene ontologies was performed on the genes that had ontology annotations using Expression Analysis Systematic Explorer (EASE) software. The number of differentially expressed genes was compared to the population of all genes on the microarray for each ontology term. A modified Fisher's test was used to calculate the EASE score, which is the upper bound of the distribution of Jackknife Fisher exact probabilities for each of the ontology terms. The ontologies were ranked by EASE score and 114 ontology terms having EASE score<0.05 are given.

FIG. 6 represents validation of gene expression by micro-arrays using Taqman quantitative real-time PCR. Quantitative real-time PCR and micro-array expression analysis of 5 genes, MMP-1, BCL2A, HEC, and CEACAM5 that were differentially expressed between ADH and ADHC and ER∃ that was not altered, are shown.

FIG. 7 represents expression of one of the most highly up-regulated genes in ADHC, mattrixmettalo proteinase I, MMP-I, in representative tissues of normal, Hyperplasia with fibrocystic change, ADH, ADHC, DCIS and Invasive breast cancer tissues by immunohistochemistry. Formalin fixed paraffin-embedded archival tissues were immunostained with antibodies against MMP-1 as described in materials and methods. Representative tissues from each category are shown (Magnification ×100). Strong staining was observed in invasive breast cancer tissues, DCIS, and ADHC samples. Staining could be seen both in the ductal epithelial cells and stroma. There was no staining in ADH tissues.

FIG. 8 represents expression of one of the most highly up-regulated genes in ADHC, Carcino Embryonic Antigen Cell Adhesion Molecule6 (CEACAM6), in representative tissues of normal, intraductal papilloma, ADH, ADHC, and Invasive breast cancer tissues by immunohistochemistry. Formalin fixed paraffin-embedded archival tissues were immunostained with antibodies against CEACAM6 as described in materials and methods. Representative tissues from each category are shown (Magnification ×100). Strong staining was observed in invasive breast cancer tissues, Fibrocystic change and ADHC samples. Staining could be seen both in the cytosol and membranes of ductal epithelial cells. There was no staining in ADH tissues.

FIG. 9 represents the ROC (Receiver Operating Characteristics) statistics of MMP-1 and CEACAM6 expression in ADH, ADHC and non-ADH benign tissues. The expression data on the above tissues for MMP-1 and CEACAM6 by IHC were analyzed individually and in combination for their Sensitivity (percentage of precancerous samples that were positive for the marker), Specificity (percentage of control precancerous samples that were negative for the marker), PPV (correctly predicting cancer development in patients who were positive for the marker) and NPV (correctly predicting non-development of cancer in patients who were negative for the marker) using S-PLUS software. Both markers have very high specificity and sensitivity and ppv values as individual markers. The sensitivity and ppv values were higher when both markers were taken together demonstrating that both markers are highly predictive of developing breast cancer and the predictive power is higher when both markers are taken together.

FIG. 10 represents expression of MMP-1 mRNA in ductal lavage samples and ADHC samples by reverse transcription and conventional PCR. The cells from ductal lavage fluid were collected by centrifugation and total RNA isolated using Qiagen RNeasy micro kit. The total RNA was reverse transcribed in a total volume of 20 micro liters to prepare cDNA. One micro liter of cDNA was used for conventional PCR with sense and anti-sense primers specific for MMP-1 in a total volume of 10 micro liters. The total 10 micro liters of PCR products were electrophoresed in agarose gels. The figure shows the PCR products generated from ADHC and Invasive breast cancer tissues and five ductal lavage samples. The absence of products in ADH and two ductal lavage samples are shown in the figure.

FIG. 11 represents micrographs of cytology of two ductal lavage samples. One sample which was diagnosed as atypia by cytology was negative for MMP-1 by both conventional PCR and quantitative real-time PCR and another was positive although it was diagnosed as benign. The figure shows that histologically diverse type of ductal cells could have cancer predictive markers such as MMP-1.

FIG. 12 represents expression of MMP-1 by reverse transcription Taqman quantitative real-time PCR in a total of forty nine ductal lavage (DL) samples, six DCIS and twelve Stage I invasive breast cancer tissues. Expression levels of five DCIS, five Stage I and all the nine positive ductal lavage samples are shown as histograms. Strong signal was observed in DCIS and Stage I tissues and a ductal lavage sample collected from a cancer patient (DL-Cancer) who had nipple discharge. Six samples diagnosed as having mild atypia were also positive. Two samples diagnosed as benign were also positive.

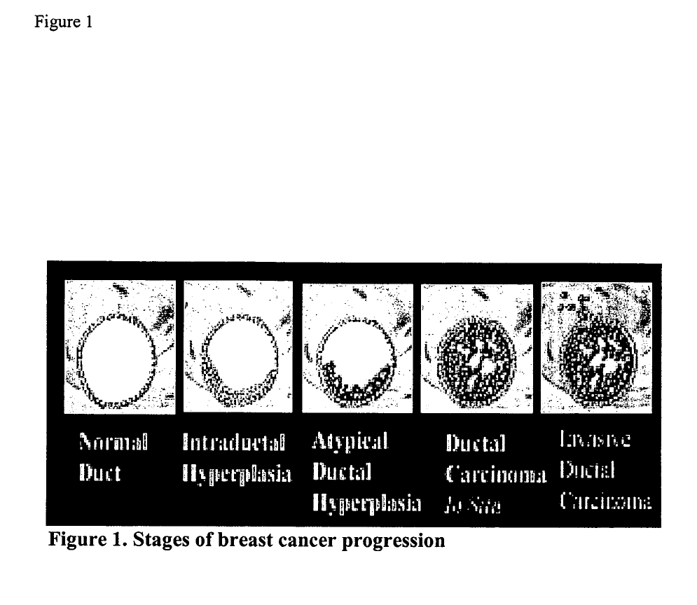

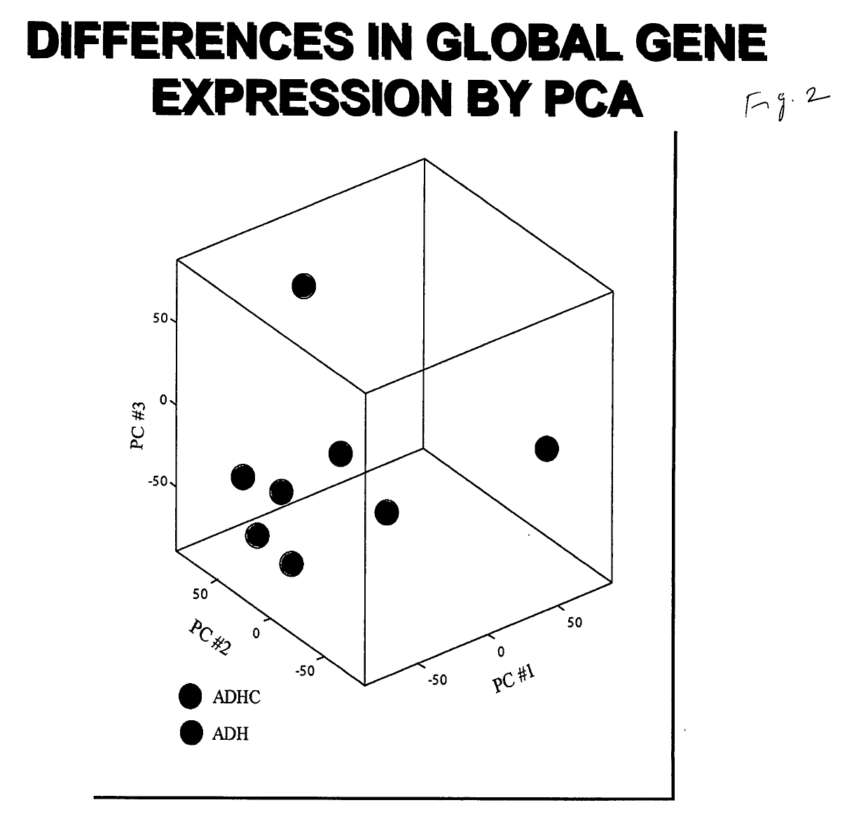

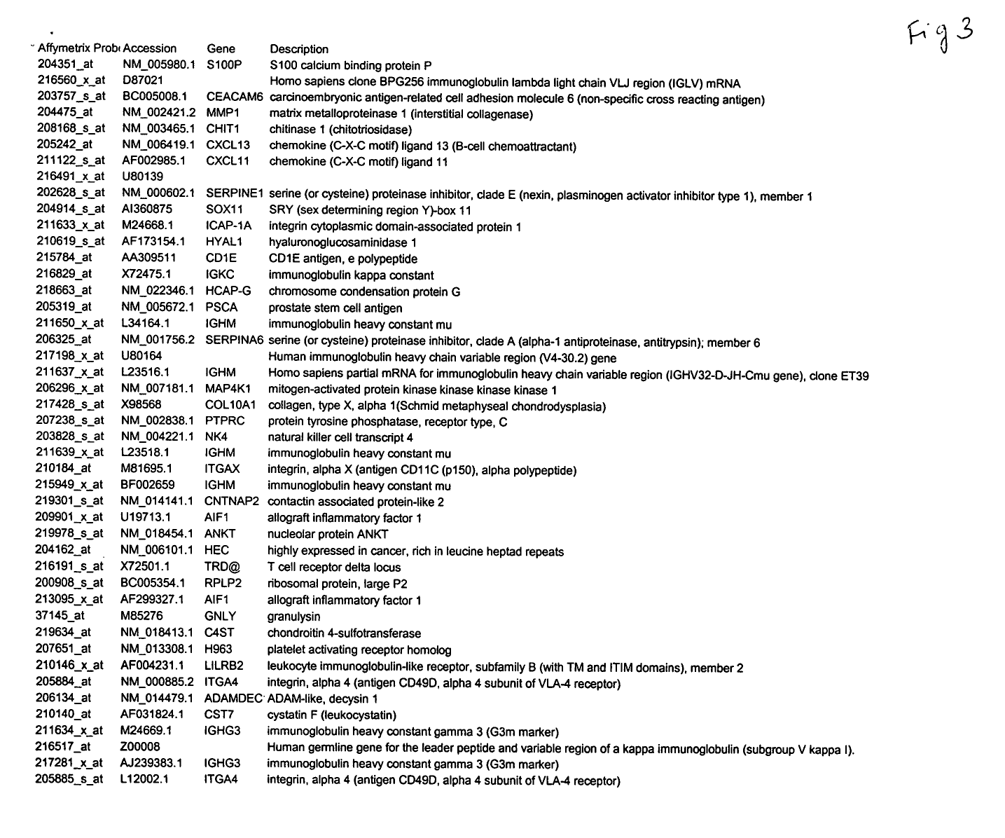

FIGS. 1, 2, 3, 4, 5, 6, 7, 8, 9, 10, 11 and 12 are seen at the end of the art.

### DETAILED DESCRIPTION OF THE INVENTION

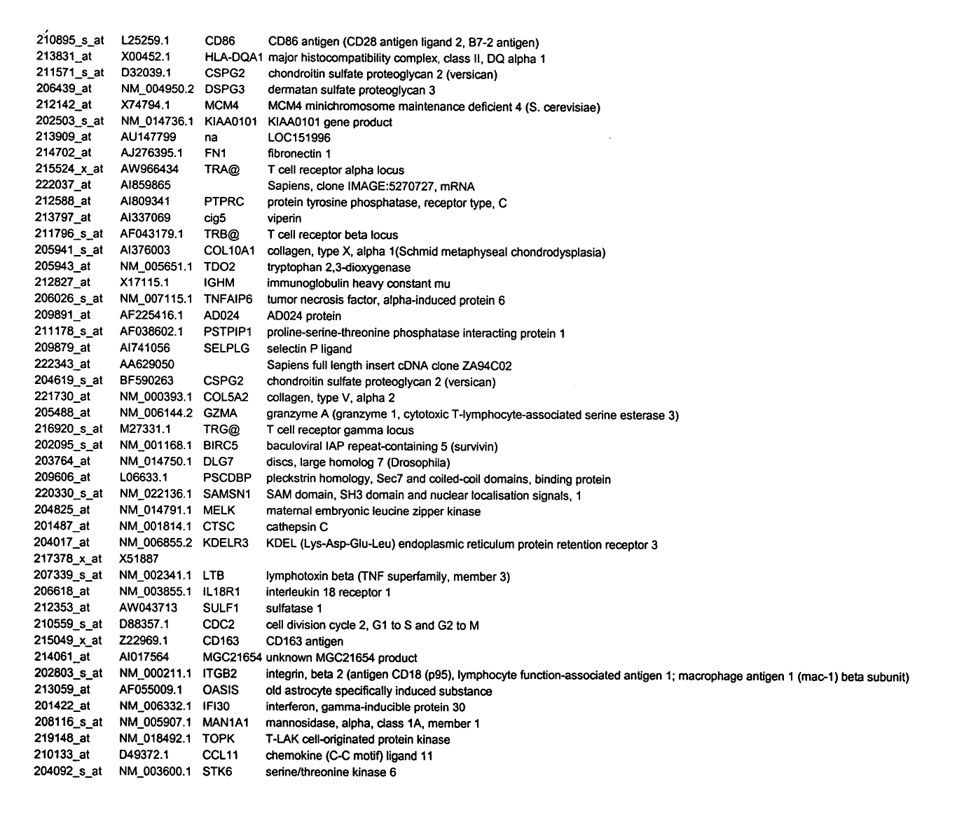

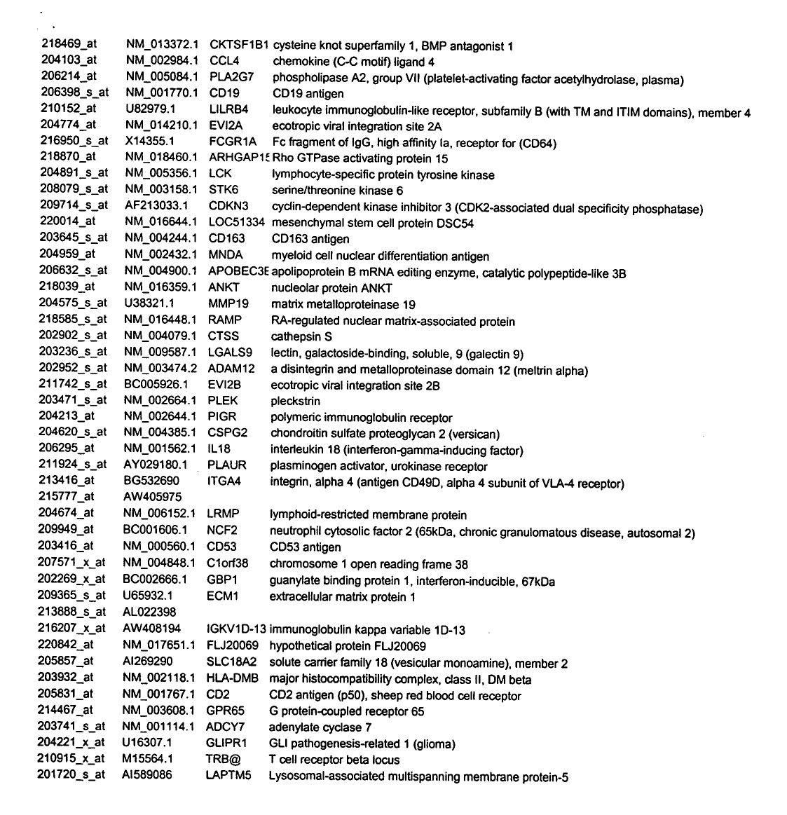

The present invention provides a selected number of genes/gene products consisting essentially of the genes/gene products selected from the group of gene numbers 1 though 541 in FIG. 3. The term “gene” as used herein is defined as polymer of DNA or RNA (i.e a polynucleotide) which can be single-stranded or double-stranded or the product of a gene, the protein (i.e. a polypeptide), synthesized or obtained from natural sources, and which can contain natural, non-natural or altered nucleotides or polypeptides. With respect to the isolated genes/gene products of the present invention, it is preferred that no substitutions, insertions, deletions, and/or inversions are present in the DNA, RNA or peptide/protein. However, it may be suitable in some instances for the isolated genes/gene products of the present invention to comprise one or more insertions, deletions, and/or substitutions.

In a preferred embodiment of the present invention, application of the expression of the selected, but not limited to, 541 genes either singly or in combination with others to detect a hyperproliferative condition or predisposition to hyperproliferative condition in a mammal.

In a preferred embodiment of the present invention, the use of the expression of the selected, but not limited to, 541 genes is either at mRNA or at protein levels.

In a preferred embodiment of the present invention, the use of the expression of the selected, but not limited to, 541 genes is at MRNA levels by Northern blot assays.

In a more preferred embodiment of the present invention, the expression of the selected, but not limited to, 541 genes is measured at MRNA levels by reverse transcription and conventional Polymerase Chain Reaction.

In a preferred embodiment of the present invention, the expression of the selected, but not limited to, 541 genes is measured at MRNA levels by reverse transcription quantitative real-time Polymerase Chain Reaction.

In a preferred embodiment of the present invention, the expression of the selected, but not limited to, 541 genes is measured at mRNA levels by cDNA expression analysis on gene micro-array chips.

In a preferred embodiment of the present invention, the expression of the selected, but not limited to, 541 genes is measured at protein levels by Western blotting method.

In a preferred embodiment of the present invention, the expression of the selected, but not limited to, 541 genes is measured at protein levels by immunohistochemistry using gene specific antibodies.

In a preferred embodiment of the present invention, the expression of the selected, but not limited to, 541 genes is measured at their protein levels by Enzyme Linked Immunosorbant (ELISA) method using protein specific antibodies.

In a preferred embodiment of the present invention, the expression of the selected, but not limited to, 541 genes, is measured at protein levels by their respective functional assays.

In a preferred embodiment of the present invention, the expression of the selected, but not limited to, 541 genes, is measured at protein levels by two dimensional gel electrophoresis followed identification by spectrophotometry such as SELDI.

In a preferred embodiment of the present invention, the expression of the selected, but not limited to, 541 genes, is measured at protein levels by antibody arrays.

The present invention includes, but not limited to, 541 genes and their products, mRNAs or proteins or peptides derived from proteins as markers, for screening the breast tissues to detect hyperproliferative condition such as precancerous cells. The invention comprises comparing the expression profiles of the isolated genes or gene products in a hyperprolific condition such as a breast disease/precancerous condition/cancer, with the normal tissue. The difference in the expression profiles of the mammal in comparison to the expression profiles of the normal control standard is indicative of a hyperproliferative condition or a benign condition or a predisposition to a hyperproliferative condition in a mammal.

The present invention includes, but not limited to, 541 genes and their products, mRNAs or proteins or peptides derived from proteins as markers, for screening a hyperprolific condition such as usual ductal hyperplasia (UDH) or atypical ductal hyperplasia (ADH) tissues to predict cancer development. The difference in the expression profiles in ADH tissues of a mammal in comparison to the expression profiles of the normal control standard is indicative of a pre-disposition to developing cancer in a mammal.

The present invention includes, but not limited to, 541 genes and their products, mRNAs or proteins or peptides derived from proteins as markers, for screening a hyperprolific condition such as atypical lobular hyperplasia (LDH) tissues to predict cancer recurrence. The difference in the expression profiles in LDH tissues of a mammal in comparison to the expression profiles of the normal control standard is indicative of a predisposed condition for developing cancer in a mammal.

The present invention includes, but not limited to, 541 genes and their products, mRNAs or proteins or peptides derived from proteins as markers, for screening a hyperprolific condition such as usual ductal hyperplasia (UDH) or atypical ductal hyperplasia (ADH) who had a history of cancer to predict cancer recurrence. The difference in the expression profiles in ADH tissues of a mammal in comparison to the expression profiles of the normal control standard is indicative of a predisposition to developing cancer in a mammal.

The present invention includes, but not limited to, 541 genes and their products, mRNAs or proteins or peptides derived from proteins as markers, for screening a hyperprolific condition such as atypical lobular hyperplasia (LDH) who had a history of cancer to predict cancer development. The difference in the expression profiles of the mammal in comparison to the expression profiles of the normal control standard is indicative of a predisposition to developing cancer in a mammal.

The present invention provides, but not limited to, 541 genes and their products, mRNAs or proteins or peptides derived from proteins as markers, for screening a hyperprolific condition such as nipple discharge to predict cancer development. The difference in the expression profiles of the mammal in comparison to the expression profiles of the normal control standard is indicative of a predisposition to developing cancer in a mammal.

The present invention provides, but not limited to, 541 genes and their products, mRNAs or proteins or peptides derived from proteins as markers, for screening a hyperprolific condition such as nipple discharge in patients who had a history of cancer to predict cancer recurrence. The difference in the expression profiles of the mammal in comparison to the expression profiles of the normal control standard is indicative of a predisposition to developing cancer in a mammal.

The present invention provides, but not limited to, 541 genes and their products, mRNAs or proteins or peptides derived from proteins as markers, for screening cells from milk ducts obtained by procedures such as ductal lavage samples or Random Periareolar Fine Needle Aspiration (RPFNA) procedures to predict cancer development. The difference in the expression profiles of disease tissue of a mammal in comparison to the expression profiles of the normal control standard is indicative of a predisposition to developing cancer in a mammal.

The present invention provides, but not limited to, 541 genes and their products, mRNAs or proteins or peptides derived from proteins as markers, for screening cells from milk ducts obtained by procedures such as ductal lavage samples or Random Periareolar Fine Needle Aspiration (RPFNA) procedures in patients who had a history of cancer to predict cancer recurrence. The difference in the expression profiles of lavage from a disease tissue the mammal in comparison to the expression profiles of the normal control standard is indicative of a predisposition to developing cancer in a mammal.

The present invention provides, but not limited to, 541 genes and their products, mRNAs or proteins or peptides derived from proteins as markers, for screening cells from milk ducts obtained by procedures such as ductal lavage samples or Random Periareolar Fine Needle Aspiration (RPFNA) procedures from subjects who had suspicious mammograms to predict cancer development. The difference in the expression profiles of the disease tissue of a mammal in comparison to the expression profiles of the normal control standard is indicative of a predisposition to developing cancer in a mammal.

The present invention provides, but not limited to, 541 genes and their products, mRNAs or proteins or peptides derived from proteins as markers, for screening a hyperprolific condition such as ductal carcinoma in situ (DCIS) to predict cancer development. The difference in the expression profiles of DCIS tissue in a mammal in comparison to the expression profiles of the normal control standard is indicative of a predisposition to developing cancer in a mammal.

The present invention provides, but not limited to, 541 genes and their products, mRNAs or proteins or peptides derived from proteins as markers, for screening a hyperprolific condition such as lobular carcinoma in situ (LCIS) to predict cancer development. The difference in the expression profiles of LCIS tissue of a mammal in comparison to the expression profiles of the normal control standard is indicative of a predisposed condition for developing cancer in a mammal.

The present invention provides, but not limited to, 541 genes and their products, mRNAs or proteins or peptides derived from proteins as markers, for screening a hyperprolific condition such as DCIS who had a history of cancer to predict cancer recurrence. The difference in the expression profiles in DCIS tissue of a mammal in comparison to the expression profiles of the normal control standard is indicative of a predisposition to developing cancer in a mammal.

The present invention provides, but not limited to, 541 genes and their products, mRNAs or proteins or peptides derived from proteins as markers, for screening a hyperprolific condition such as LCIS who had a history of cancer to predict cancer recurrence. The difference in the expression profiles LCIS disease tissue of a mammal in comparison to the expression profiles of the normal control standard is indicative of a predisposition to developing cancer in a mammal.

The present invention provides, but not limited to, 541 genes and their products, mRNAs or proteins or peptides derived from proteins as markers, for screening a hyperprolific condition in a mammal who had a history of any breast lesion or cancer to predict cancer development/recurrence. The difference in the expression profiles in a disease tissue of a mammal in comparison to the expression profiles of the normal control standard is indicative of a predisposition to developing cancer in a mammal.

The present invention provides, but not limited to, 541 genes and their products, mRNAs or proteins or peptides derived from the proteins as markers, for screening blood from a mammal to predict cancer development or a pre-disposition to cancer development. The difference in the expression profiles in the blood of a mammal in comparison to the expression profiles of the normal control standard is indicative of a predisposition to developing cancer in the mammal.

The present invention provides, but not limited to, 541 genes and their products, mRNAs or proteins or peptides derived from proteins as markers, for screening blood from a mammal who had a history of breast cancer to detect precancerous condition and predict breast cancer recurrence. The difference in the expression profiles of the blood of a mammal in comparison to the expression profiles of the normal control standard is indicative of a predisposition to developing cancer in a mammal.

The present invention provides, but not limited to, 541 genes and their products, mRNAs or proteins or peptides derived from proteins as markers, for screening blood from a mammal to predict cancer development. The difference in the expression profiles of the mammal in comparison to the expression profiles of the normal control standard is indicative of a predisposition to developing cancer in a mammal.

The present invention provides, but not limited to, 541 genes and their products, mRNAs or proteins or peptides derived from proteins as molecular targets either singly or in combination with others for designing drugs to treat a hyperproliferative or precancerous or a benign condition or cancerous condition in a mammal.

The present invention provides, but not limited to, 541 genes and their products, mRNAs or proteins or peptides derived from proteins as molecular targets either singly or in combination with others for designing drugs to treat a hyperproliferative or precancerous or a benign condition or cancerous condition of the breast in a mammal.

The present invention provides, but not limited to, 541 genes and their products, mRNAs or proteins or peptides derived from proteins as molecular markers either singly or in combination to follow up response to a therapeutic treatment for a hyperproliferative or precancerous or a benign condition or cancerous condition in a mammal.

The present invention provides, but not limited to, 541 genes and their products, mRNAs or proteins or peptides derived from proteins as molecular markers either singly or in combination to follow up response to a therapeutic treatment for a hyperproliferative or precancerous or a benign condition or cancerous condition of the breast in a mammal.

The present invention provides, but not limited to, 541 genes and their products, mRNAs or proteins or peptides derived from proteins as molecular markers either singly or in combination to screen therapeutic drugs in vitro for their effectiveness in treatment of cancer, such method comprising treatment of tissue culture cells in vitro or in animals carrying hyperproliferative cells and determining gene expression to assess effect of drug treatment or in response to a therapeutic treatment for a hyperproliferative or precancerous or a benign condition or cancerous condition of the breast in a mammal.

The present invention provides, but not limited to, 541 genes and their products, mRNAs or proteins or peptides derived from proteins as molecular markers either singly or in combination to design vaccines to prevent a hyperproliferative or precancerous or a benign condition or cancerous condition in a mammal.

The present invention provides, but not limited to, 541 genes and their products, mRNAs or proteins or peptides derived from proteins as molecular markers either singly or in combination to design vaccines to prevent a hyperproliferative or precancerous or a benign condition or cancerous condition of the breast in a mammal.

In a preferred embodiment of the present invention, the hyperprolifertive condition is cancer, benign tumors, fibroids, polyps, cysts and the like. In a more preferred embodiment, the hyperproliferative condition is the hyperproliferation of the breast, ovary, uterus, bone, brain, female genital tract, male genital tract, gastrointestinal tract, nervous system, immune system, testis or prostrate or any other part of a mammal.

The following examples further illustrate the invention but, of course, should not be constructed as in any way limiting its scope.

### EXAMPLES

Definitions. Ductal lavage, Cells removed from the milk ducts of breasts by inserting a thin flexible catheter into the milk duct opening in the nipple under local anesthesia and injecting normal saline and gently pushing the fluid to flush out the loose cells in the entire duct; RPFNA, This is an invasive procedure to obtain samples of cells by fine needle aspiration. Typically, eight to 10 aspirations are performed per breast, hakf through the upper quadrant site and half through the upper inner quadrant site after numbing the breast with lodocaine. The cells obtained by this procedure have been used to assess for any abnormalities similar to ductal lavage (Fabian, et al, J. Natl. Cancer Inst. 92, 1217-1227 (2000)); Hyperproliferation, normal looking cells dividing and expanding at a fast rate; Benign, A condition where cells look normal but dividing at a fast rate forming several layers in some instances tumors; Atypical ductal hyperplasia (ADH), a condition where the cells and their nuclei look abnormal but there are no cancerous cells, ADH from patients who developed cancer concurrently or subsequently (ADHC), Ductal Carcinoma In Situ (DCIS), in this condition, the cancer cell growth is restricted to the lumen of the duct but not invaded the stroma; Lobular Carcinoma In Situ (LCIS), in this condition, the cancer cell growth is restricted to lobule or the ends of ducts; and IBC, Invasive Breast Cancer.

Abbreviations. ADH, Atypical Ductal Hyperplasias from patients who had no prior history of cancer and did not develop cancer in 5 years after diagnosis of ADH; ADHC, Atypical Ductal Hyperplasias from patients who had cancer either simultaneously, or had cancer before or developed subsequently after the diagnosis of ADH; IBC, Invasive Breast Cancer; DCIS, Ductal Carcinoma In Situ; LCIS, Lobular Carcinoma in Situ; MMP-1, Matrix Mettalo proteinase I. CEACAM6, Carcino Embryonic Antigen Cell Adhesion Molecule 6; PCR, Polymerase Chain Reaction, and GAPDH, Glyceraldehyde 3-Phosphate dehydrogenase.

Materials:

Fresh ADH and ADHC tissue and breast cancer tissue samples from patients undergoing breast surgeries were collected immediately after the diagnosis was made, and stored at −80° C. until use. Tissue samples for research were routinely harvested immediately adjacent to the histologic/diagnostic section and were considered to be representative of the tissue utilized for diagnosis. Breast cancer predictive molecular markers were identified from ADHC tissues against a control group who did not have cancer five years either before or after diagnosis of ADH by global gene expression analysis on U133A Human GeneChip (Affymetrix). A total of six ADHC tissues were used for Micro-array analysis and a total of ten tissues with no history of breast cancer for five years pre-/post diagnosis of ADH were used as control group. Micro-array analyses were performed either on individual or pooled RNA samples. In the case of ADHC, three individual RNA samples and one pooled sample from three different patient tissues were analyzed (Table 1).

TABLE 1
ADHC SAMPLES USED FOR cDNA MICRO-ARRAYS

| | | | ADHC Diagnosis | |
|---|---|---|---|---|
| | ADHC | Cancer | (Years Pre-/Post | |
| | Diagnosed | Diagnosed | Diagnosis of | Type of Surgery |
| # | Breast | Breast | Cancer) | for Cancer |
| 1 | Left | Left | Simultaneous | Partial Mastectomy |
| 2 | Left | Right | 2 Years Post | Lumpectomy |
| 3 | Right | Right | 1 Year Pre | Modified Radical |
| | | | | Mastectomy |
| *4 | Right | Right | 5 Years Post | Partial Mastectomy |
| *5 | Right | Left | 1 Year Post | Modified Radical |
| | | | | Mastectomy |
| *6 | Right | Right | 14 Months Post | Mass Excision |

*Pooled for analysis

For ADH samples, four analyses were performed. For one sample run, RNA from three different patient tissues were pooled, for a second sample, RNA from four different patient tissues were pooled, third run was performed with RNA pooled from two different patient tissues and a fourth run from one individual patient tissue. Whenever RNA samples were pooled, equal amounts from each patient tissue were pooled. The small amounts of tissue obtained in some cases necessitated pooling of RNA samples for micro-array analyses. Information about the diagnosis of cancer pre-/post diagnosis of ADHC and ADH were obtained from Tumor Registry data bases, surgical pathology data bases, and follow up visits with surgical-, and clinical-oncologists. Ductal lavage samples were collected using InDuct™ Breast MicroCatheters obtained from Cytyc Health by a breast surgical oncologist and all the samples were examined by cytopathologist.

Methods:

Total RNA from frozen breast tissues was extracted using Trizol reagent (Gibco-BRL Life Technologies) as described (Poola, I. et al Cancer. 94: 615-623 (2002), Poola, I. et al J. Steroid Bichem. Mol. Biol. 82, 169-179 (2002)). RNA isolations from ductal lavage samples were performed using Qiagen RNeasy Micro total RNA isolation columns. RNA integrity was verified by both electrophoresis in 1.5% agarose gels and amplification of the constitutively expressed gene, glyceraldehyde-3 phosphate dehydrogenase (GAPDH). For cDNA gene array analysis, total RNA was further purified on two sequential Qiagen RNeasy Mini total RNA isolation columns according to the manufacturer's protocol.

For gene microarray analysis, purified total RNA samples were processed for oligonucleotide micro-array analysis according to the protocols specified by Affymetrix. Briefly, first and second strand cDNA was synthesized from 7:g of total RNA using Superscript Choice System double stranded cDNA synthesis kit (Invitrogen) with a HPLC-purified oligo dT 24 primer containing a T7 RNA polymerase promoter sequence at it's 5′-end (Affymetrix, Santa Clara, Calif.). After the second strand synthesis, cDNA was extracted using a cleanup column from Qiagen. The double stranded cDNA was used as a template to synthesize biotin-labeled cRNA by in vitro transcription using a Bioarray High Yield RNA Transcript Labeling Kit (Enzo Diagnostics, Farmingdale, N.Y.) according to the manufacturer's instructions. Biotin-labeled cRNA was purified on a RNeasy Mini column.

Twenty micrograms of biotin-labeled cRNA were fragmented to an average size of 35-200 nucleotides by heating at 94° C. for 35 min with 1× fragmentation buffer (40 mM Tris-acetate pH 8.1, 100 mM KoAc, 30 mM MgoAC) in a final volume of 40:1. The fragmented cRNA (15 :g) was added to the hybridization buffer (100 mM MES, 1M [Na+], 20 mM EDTA, 0.01% Tween 20), herring sperm DNA, acetylated BSA, and hybridization controls to complete the hybridization solution. The controls were bacterial and phage cRNA that served as internal controls for hybridization efficiency. The cRNAs were hybridized to U133A Human GeneChip (Affymetrix) that represents 22,283 biological oligonucleotides including normalization controls. After hybridizing in a rotisserie oven for 16 hours at 45° C., the arrays were developed with R-Phycoerythrin streptavidin (10 :g/ml, Molecular Probes) and amplified with Goat IgG and biotinylated anti-streptavidin antibody (Vector Laboratories) using Affymetrix Fluidics station. Arrays were washed with a non-stringent buffer (20×SSPE, 10% Tween 20) and a stringent buffer (12×MES, 5M NaCl, 10% Tween 20). The arrays were then scanned on an Agilent GeneArray Scanner. Fluorescence intensities on scanned images were quantified, correlated for background noise and normalized to a standard expression level, and then exported to GeneSpring (Silicon Genetics) for further analysis. Affymetrix Microarray Suite software calculates a % positive of present genes.

For the purpose of conventional PCR and Taqman quantitative real-time PCR, cDNA was prepared from isolated total RNA by reverse transcription using Omniscript reverse transcriptase as described (Poola, I et al FEBS Letters, 516:133-138 (2002)). Briefly, the RNA was denatured by heating for 3 minutes at 65° C., cooled on ice, and incubated with reverse transcriptase reaction mixture. The standard mixture contained one microgram of total RNA, 10 U of RNAse inhibitor, 0.5 mM each of dNTPs, 1-:M random hexamers and 4 U of Omniscript reverse transcriptase in a total volume of 20:1. For reverse transcription, tubes were incubated at 37° C. for 60 min, followed by 95° C. for 5 min and finally rapidly cooled. Conventional PCRs were conducted as described (Poola, I et al FEBS Letters, 516:133-138 (2002)) Quantitative real-time PCR was performed using Assays-on-Demand™ Gene Expression reagents from Applied Biosystems in GeneAmp ABI Prism 7900HT Sequence Detection System at 50% ramp rate as described (Poola, I Anal. Biochem. 314: 217-226, Poola, I. Endocrine, 22: 101-111 (2003).

For unsupervised analysis of micro-array data, the probes having signal detection p<0.065 (Present) in a minimum of 50% arrays were selected. Principal component analysis (PCA) optimizing correlations on about 12,000 probes indicated 62% of variance is accounted by the first three principal components (Mandia, K. V. et al Multivariate Analysis (1975), London: Academic Press). The analysis was computed using software, Partekpro 5.0, Partek, Inc. (St. Charles, Mo., 63304). In order to identify differentially expressed genes between ADH and ADHC, logarithmic ratios (to the base 2) of ADHC/ADH were calculated by comparison analysis of MAS5 for all 16 combinations of ADHC and ADH pairs. The ratios associated with a “change” p-value<0.003 (increase) or >0.997 (Decrease) or a signal detection p-value<0.065 were included for further calculations. Mean of all ratios, one sample t-test p-value, number of Increase, and Decrease calls and sample size were calculated for each gene. Differentially expressed genes between ADHC and ADH samples were selected when mean ratio is 2-fold, p<0.001, at least 8 of the 16 ratio values were present and there was no tie between number of Increase and Decrease calls. Over-representation analysis of gene ontologies was performed on the genes that had ontology annotations using Expression Analysis Systematic Explorer (EASE) software (Hosack, D. A. et al, Genome Biol. 4, R70 (2003) The number of differentially expressed genes was compared to the population of all genes on the microarray for each ontology term. A modified Fisher's test was used to calculate the EASE score, which is the upper bound of the distribution of Jackknife Fisher exact probabilities for each of the ontology terms. The ontologies were ranked by EASE score and 114 ontology terms having EASE score<0.05.

The relative expression patterns of genes having highest expression change are shown by hierarchical clustering (Eisen, M. B. et al Proc. Natl. Acad. Sci U.S.A, 95, 14863-14868 (1998)) Hierarchical clustering of expression of 75 genes (35 top highly up-regulated and 35 bottom highly down regulated plus five other interesting genes) using 1-ρ as distance metric, where ρ is Pearson correlation coefficient. Expression values were mean centered for each gene indicating relative differences between ADHC and ADH from the mean. The cluster is color coded using red for up-regulation and green for down-regulation.

For Immunohistochemical (IHC) detection of cancer predictive markers, unstained paraffin-embedded atypical ductal hyperplastic tissue sections were immuno-stained for two of the most highly up-regulated markers, matrix mettalo proteinase I, MMP-1 and CEACAM6 using monoclonal antibodies from Santa Cruz Biotechnologies (Santa Cruz, Calif.) and BioGenex respectively. Briefly, slides were deparaffinized in 2 changes of toluene for 5 minutes each and gradually re-hydrated through five changes of graded EtOH (100%, 90%, 70%, 50%, 30%, and distilled water, 2 min. each). Antigens were unmasked by treating the slides in a steamer for 25 min in 10 mM citrate buffer pH 6.0. Tissue sections were cooled and blocked with 3% H₂O₂ in methanol for 20 min and washed with PBS. The slides were incubated with monoclonal antibodies against MMP-1 (Santa Cruz Biotechnologies) or CEACAM6 (Biogenex). The slides were rinsed and incubated with EnVision peroxidase conjugated secondary antibody (Dakocytomation, Canada) for 30 min. The slides were washed and incubated with peroxidase substrate (DAB liquid chromogen, from Dakocytomation, Canada) for 5 min. Finally, the slides were washed and stained with Haematoxylin, mounted and visualized under Leica DMRXA microscope. All slides and micrographs for the above markers were evaluated by two pathologists to assess the presence of these two markers. A total of 108 tissue samples were stained for each of MMP-1 and CEACAM6 in duplicate by the above procedure. To ensure that the slides used for staining with antibodies contained only the ADH/ADHC tissues but not cancer or benign tissues, the first and the last section cut from each paraffin block were stained with Hematoxylin-Eosin and visualized under microscope and those slides that had only atypical cells were used.

Results:

### Example 1

This example shows that gene expression in ADHC is significantly different from ADH tissues and several genes are altered significantly in ADHC tissues. To identify the differentially altered genes, first, the gene expression profiles of ADH and ADHC were analyzed by unsupervised Principal Component Analysis (PCA) using signals of all the expressed genes (Table 2) and after removing absent calls and genes with 50% missing data.

TABLE 2
Summary of number of genes called present in the samples analyzed
by Affymetrix Microarray Suite (MAS) ver. 5 Software

| Sample | Number of genes | Percent Present and |
|---|---|---|
| Name | present and Marginal | Marginally Present |
| ADH1 | 11,539 | 52% |
| ADH2 | 12,116 | 54% |
| ADH3 | 12,829 | 58% |
| ADH4 | 12,511 | 56% |
| ADHC1 | 12,056 | 54% |
| ADHC2 | 10,849 | 49% |
| ADHC3 | 11,897 | 53% |
| ADHC4 | 12,184 | 54% |

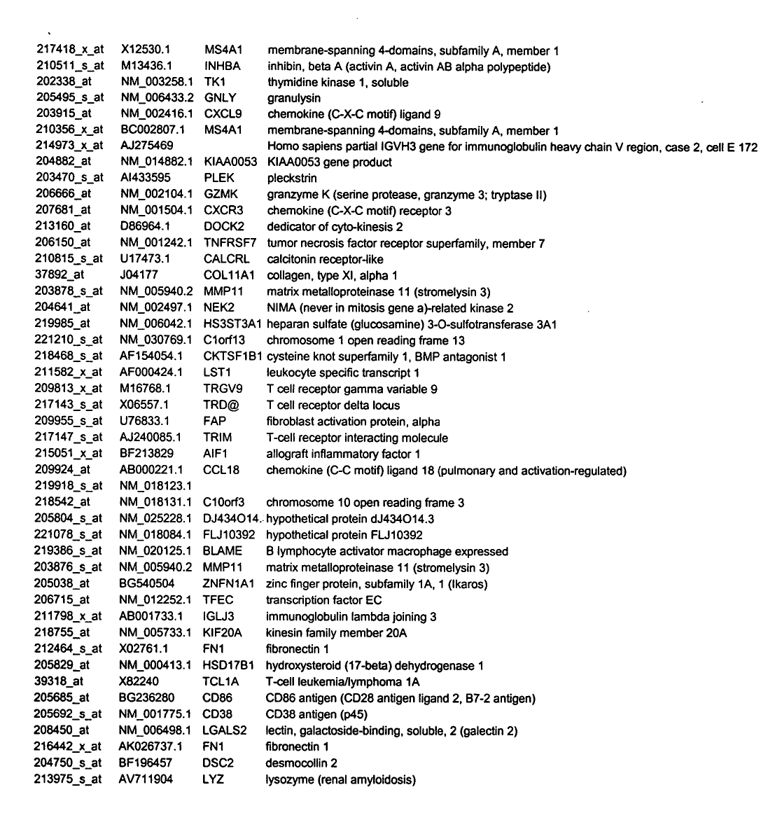

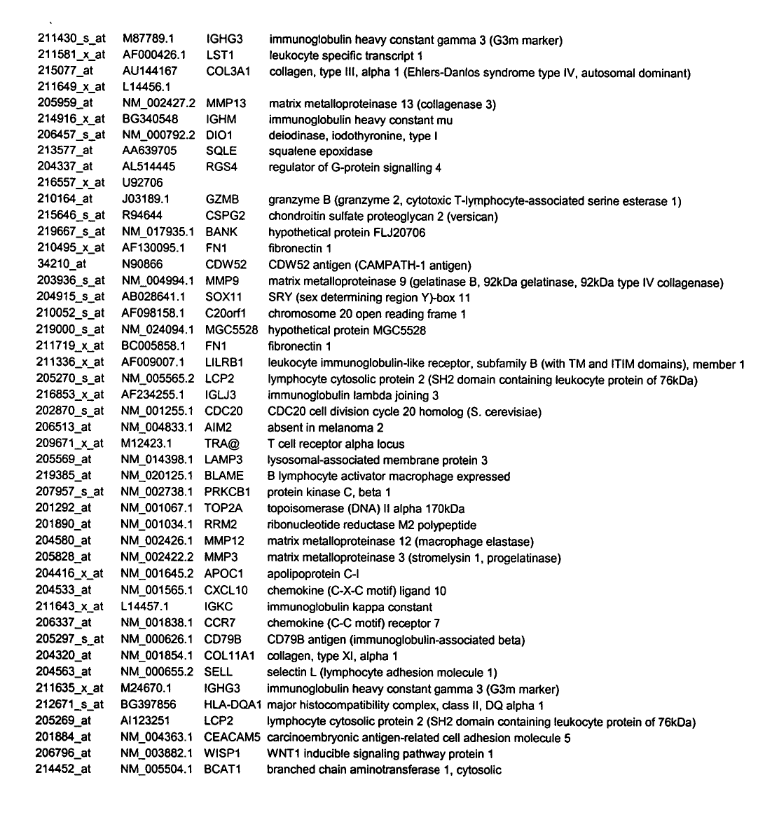

A projection on three principal components accounting for 62% of the variance revealed clear segregation between ADH and ADHC arrays as shown in FIG. 2, indicating that the global gene expression patterns of these two groups are significantly different. This observation also demonstrated that differences between ADH and ADHC were sufficient to cluster these into two groups. Additionally, the PCA revealed that ADHC samples were more diverse than the ADH samples suggesting that their underlying biology may be different.

To determine the statistical significance of gene expression differences between ADH and ADHC for each gene by one sample t-test using ADHC/ADH logarithmic ratios were used. Differentially expressed genes between ADHC and ADH samples were selected when the mean ratio was 2-fold, p<0.001, at least 8 of the 16 ratio values were present and there was no tie between number of Increase and Decrease calls. At this statistical significance level, a total of 540 gene probes differed significantly between the two groups (FIG. 3). Of the 540 probes, 371 were elevated at mean ratio of 2-fold up and 169 are down regulated. These data demonstrate that the biology between the two groups is significantly different.

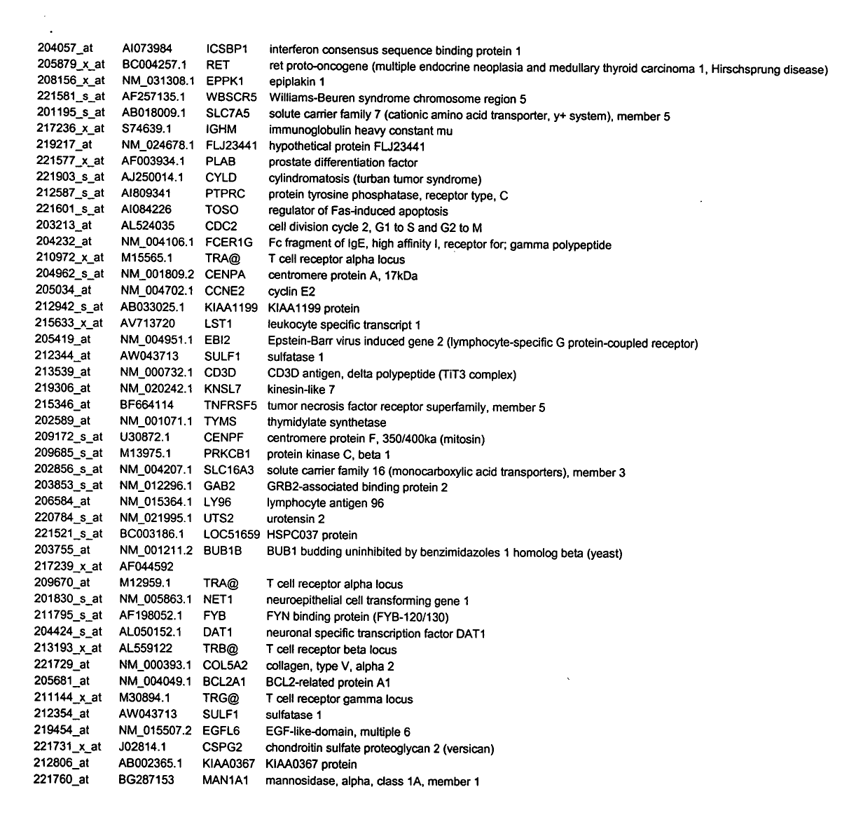

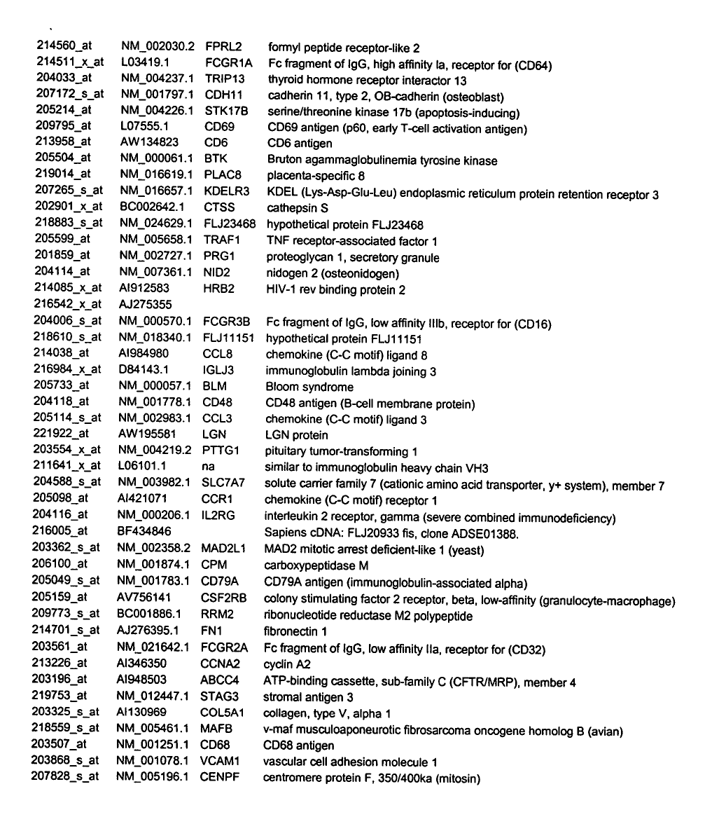

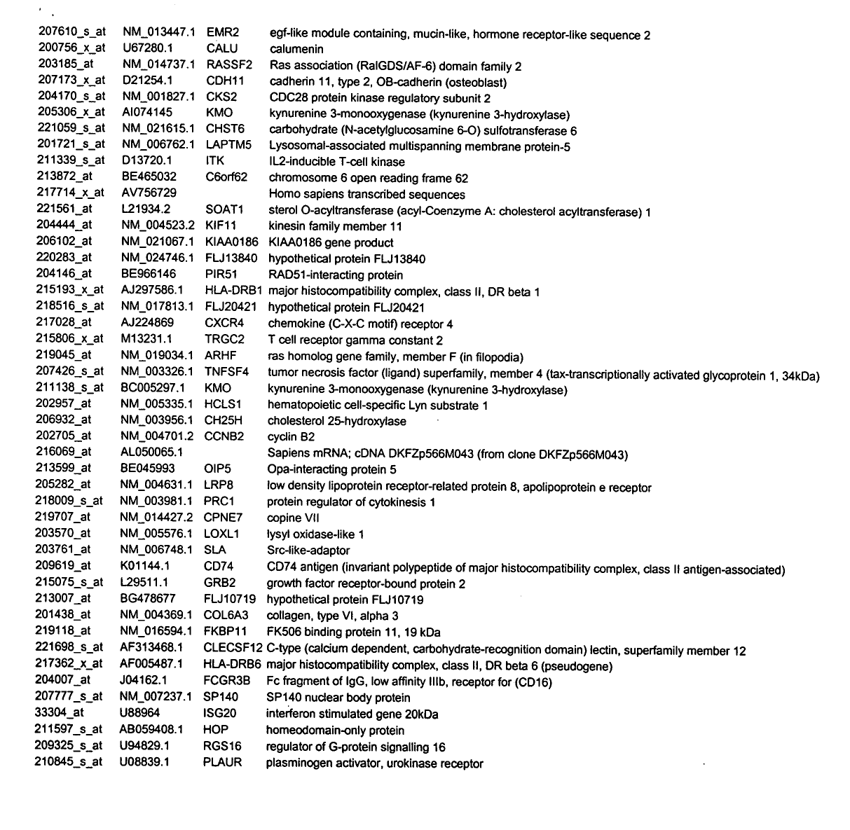

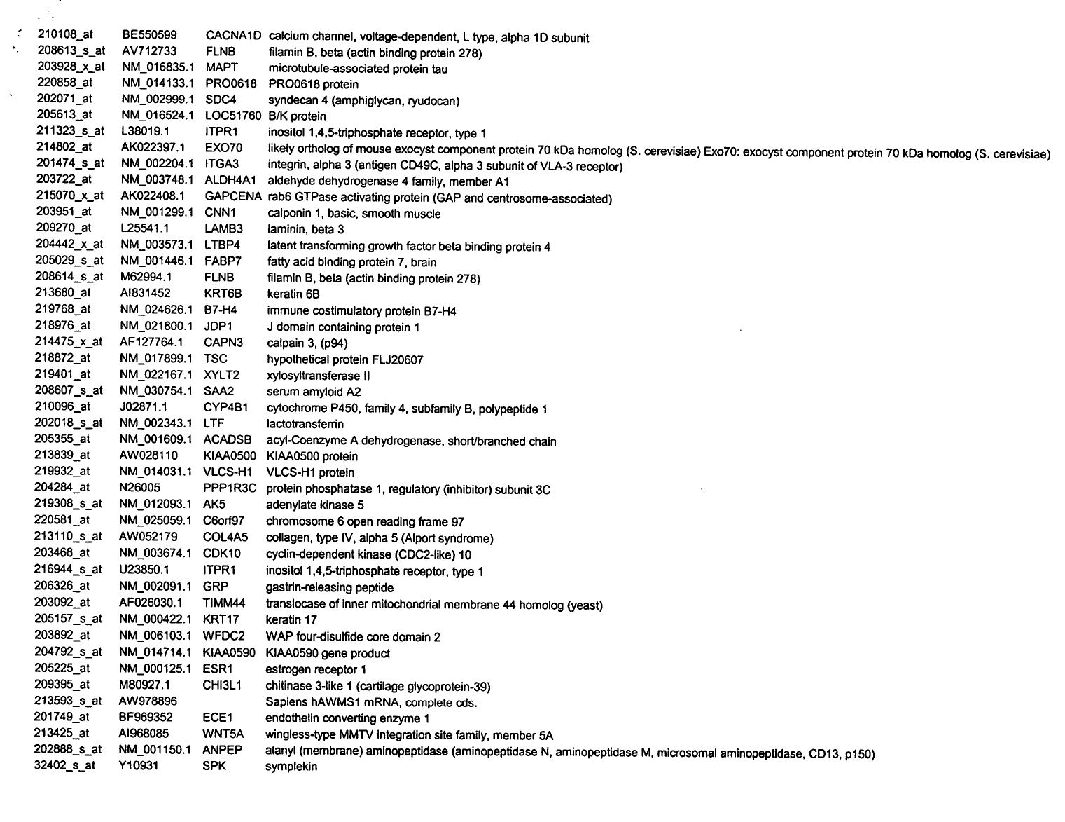

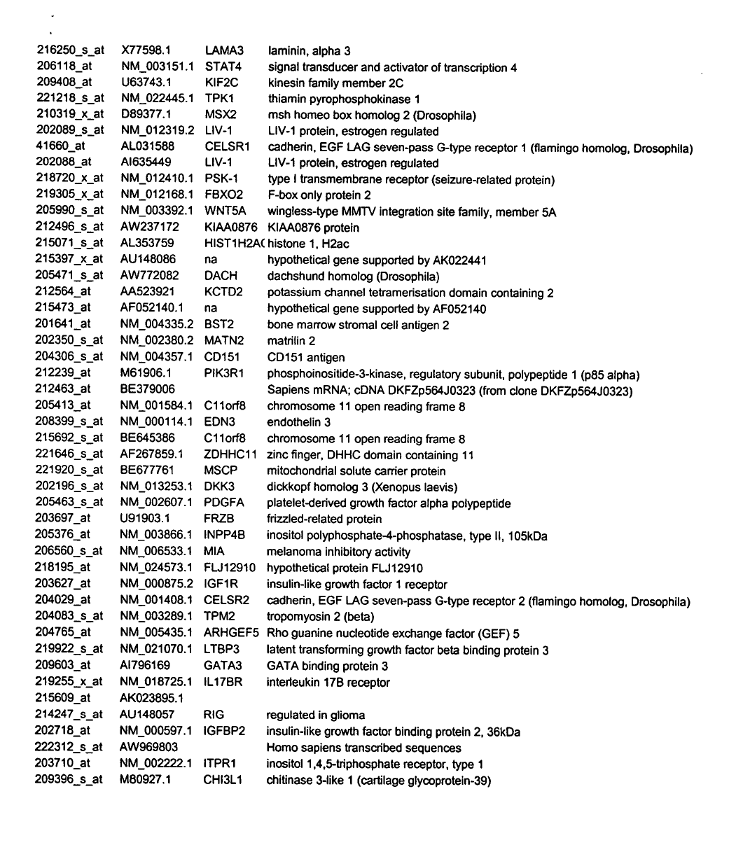

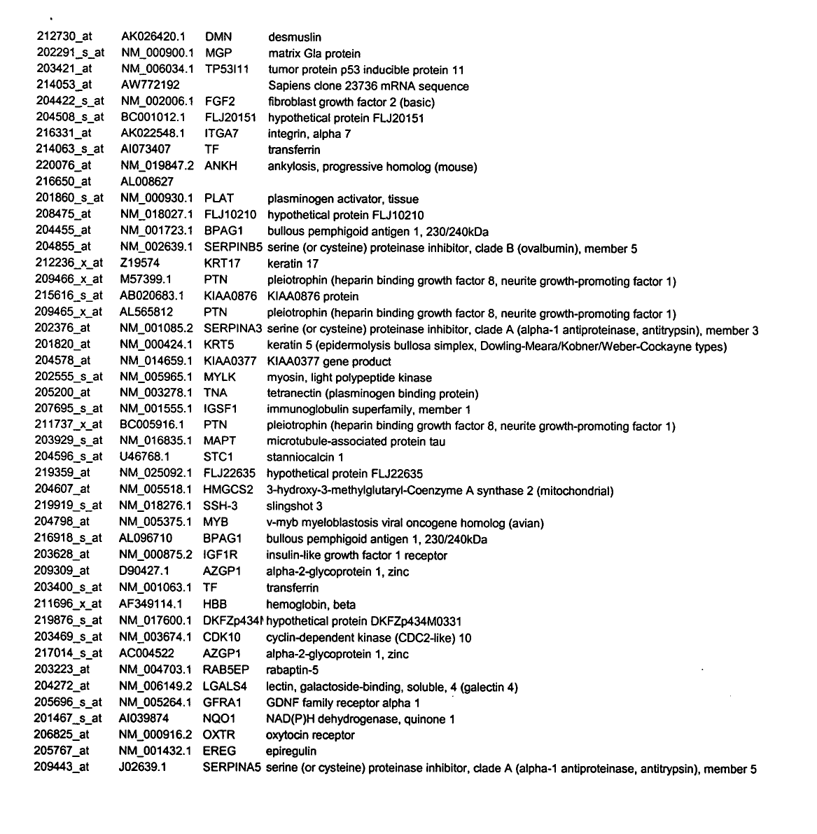

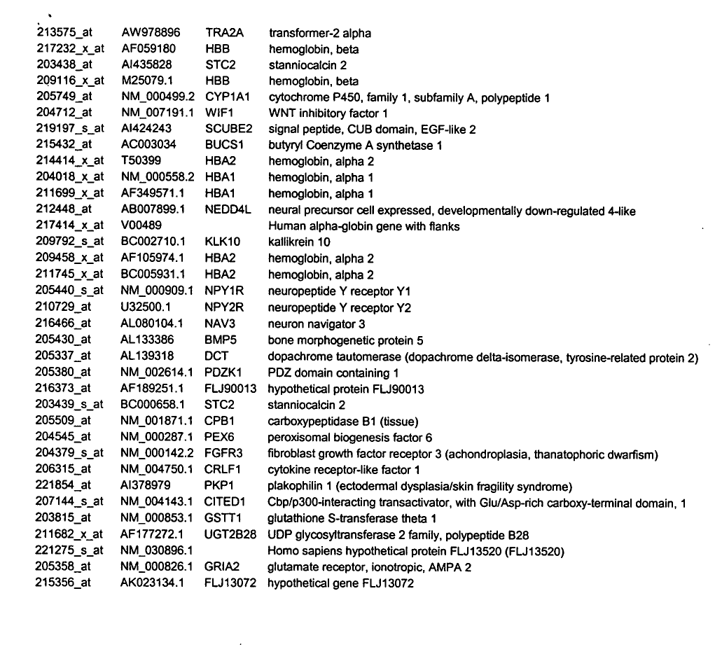

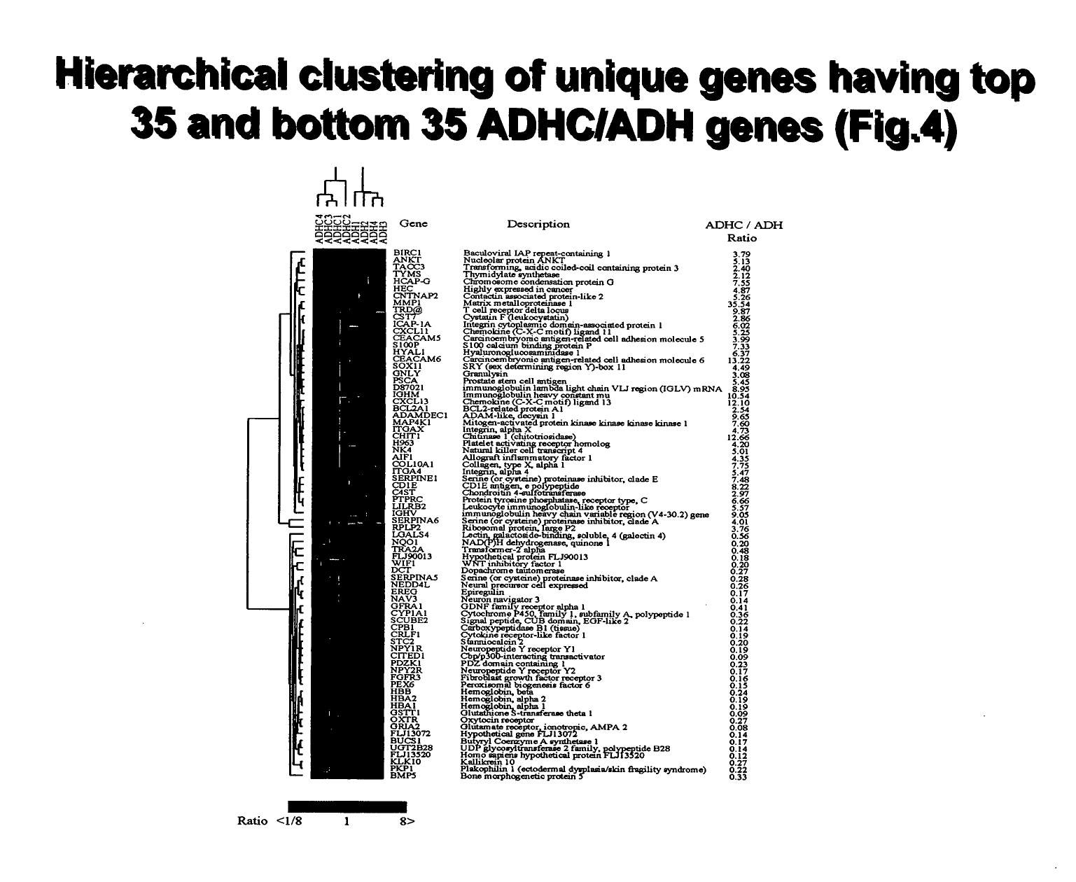

Hirarchial clustering of unique genes having top 35 and bottom 35 ADHC/ADH ratios and 5 interesting genes (BCL.2A1, BIRC1, TACC3, CEACAM5, and TYMS) by average linkage and centered correlation is shown in FIG. 4. Logarithmic signal values were mean centered for each gene. Each point in space represents one array. The cluster is color coded using red for up-regulation and green for down regulation and black for medium expression.

To further understand the differences in biology, ontologies of these genes were analyzed by Expression Analysis Systematic Explorer. Interestingly, a large number of over-represented ontologies were identified by comparing these genes to the population on array (FIG. 5).

A number of genes that were implicated in breast cancer previously (Lacroix, M. et al The International J. Biological Markers. 17, 5-23 (2002), for example CEACAM5, several MMPs, TYM5, MAD2, are seen in this list (FIG. 3). In addition, ten (ANKT, CENPA, TOPK, RRM2, TOP2A, NEK2, CDKN3. BUB1, BIRC5 AND CKS2) of the thirty eight genes identified by Xiao-Jun et al (Xiao-Jun Ma. et al. Proc. Natl. Acad. Sci. USA, 100, 5974-5979 (2003)) and shown to be altered during breast cancer progression were also seen in FIG. 3. Interestingly, the ER∀, that mediates actions of the allegedly breast cancer causing estrogen (Gruber, C. J. et al New England J. Med. 346, 340-352 (2002) and Harris, R. et al New England J. Med. 327, 390-395, (1992)), and shown to be increased in some breast cancers (Leygue, E. et al Cancer Res. 58, 3197-3201 (1998)) was not altered in ADHC samples.

### Example 2

This example shows how micro-array data is verified by Taqman quantitative real-time PCR. To verify micro-array data, the expression of a set of 5 genes, MMP-1, CEACAM5, BCL2A, ER∃, and HEC were examined in a total of ten ADH, and six ADHC samples by reverse transcription quantitative real-time PCR. These genes were chosen because of their functional significance and their expression in breast cancer tissues have been well established (Lacroix, M. et al The International J. Biological Markers. 17, 5-23 (2002)). With an exception of ER∃, the expressions of all other four genes were significantly elevated in ADHC samples by micro-array analysis. Profiling their expression by quantitative real-time PCR showed excellent concordance with the micro-array data (FIG. 6). Quantitative real-time PCR validation clearly established the authenticity of micro-array data obtained by global gene expression analysis on Affymatrix platform.

### Example 3

This example shows that the genes that were differentially altered in ADHC tissues regulate several cellular processes (Poola et al, Nature Medicine, 11, 481-483, 2005). Based on the differentially expressed genes in ADHC, some of the cellular processes that are altered in ADHC tissues are shown in Table 3 and described below. Briefly, the ADHC tissues have:

TABLE 3
Cellular processes deregulated in ADHC tissues

| | | Genes Significantly |
|---|---|---|
| Cellular Process | Genes Significantly Up-Regulated | Down-Regulated |
| Cell Cycle Check | Cyclin A, Cyclin E, Cyclin B, CDC2, | None |
| Points | NEK2, HCAPG, CENPA, CENPF, | |
| | MAD2L1, BUB1, PTTG1, CDC20, ANKT, | |
| | HEC, KIF2C, RAMP, PRC1, DOCK2, | |
| | KIF20A | |
| Nucleic Acid | TK1, RRM2, and Thymidylate Synthase | AK5 |
| Biosynthesis | | |
| Estrogen Metabolism | HSD17B1 | UGT2B23 |
| Cell-Cell and Cell- | MMP-1, MMP-3, MMP-9, MMP-11, MMP- | SERPINA-3, and -5 |
| ECM Interactions | 12, MMP-13, MMP-19, PLAUR (Urokinase | |
| | receptor) and Cathepsin C | |
| Cell Surface Polarity | CEACAM5, CEACAM6, Galectin 5, | CELSR-1, and -2, |
| and Architecture | Galectin-9, CDH11, DSC2 and VCAM1 | |
| Signal Transduction | GRB2, MAP4K1, TOPK, T-LAK, STK6, | LTBP3-, and -4, |
| | STK17B, EGFL6 and PRKCB1 | IGFBP2, |
| Proto-Oncogenes | RET1, NET, WISP1 and MAFB, | None |
| Tumor Suppressors | None | WIF1, FRZB |
| Apoptosis | BCL2A1 | None |
| Detoxification | None | GST-transferase, |
| | | CYP4B1 and CYP1A1 |
| Lymphocytic | Over 100 genes | None |
| Infiltration | | |

Very high mitotic Index. A total of nineteen cell cycle checkpoint genes that regulate every phase of cell cycle and were previously shown to be up regulated in several cancers including breast were significantly increased in ADHC tissues. Two genes, Cyclin A and Cyclin E that play a fundamental role in the transition of G1/S phase (Pagano, M. et al EMBO J. 11, 961-71 (1992) and Koff, A. et al Science, 257, 1689-1694 (1992)) by interacting with CDK2, and three genes, Cyclin B (Hwang, A. et al J. Biol. Chem. 273, 31505-31509 (1998)), CDC2 (Andez et al The EMBO J. 17, 470-481 (1998)) and NEK2 (O'Connel et al Trends in Cell Biology, 13, 221-228 (2003)) that promote the transition from G2 to M Phase were significantly increased. Three genes, chromosome condensation protein G (Kimura, K et al J. Biol. Chem. 276, 5417-5420 (2001)) CENPA (Howman E. V et al Proc. Natl. Acad. Sci. 97, 1148-53 (2000)) (centromere protein A, 17 kDa), and CENP (Kitagawa, K and Kieter, P, Nature Reviews., Mol. Cell. Biol. 2: 678-87)) that regulate condensation and segregation of chromosomes in prophase were also elevated. Seven proteins that regulate metaphase to anaphase transition MAD2L1 (Li, Y et al, Science, 274:246-248 (1996)), BUB1 (Onyang, et al Cell Growth and Differentiation, 9: 877-885 (1998)), PTTG1 (Zou, H et al, Science, 285:418-422 (1999)), CDC20 (Weinstein, J. J. Biol. Chem. 272:28501-28511 (1997)), ANKT (Duensing, S et al, Crit. Re. Eucaryot. gene. Exp. 13, 9-23 (2003)), HEC (Martin, S. et al Science 297: 2267-2270 (2002)), and KIF2CV (Kim, I. G et al Biochem. Biophys. Acta, 1359, 181-186 (1997)) and four proteins that regulate splitting of cytoplasm, RAMP (Cheung, W. M et al J. Biol. Chem, 276:17083-91 (2001)) PRC1 (Molinari, C et al J. Cell. Biol. 157: 1175-1186 (2002)) DOCK2 and KIF20A (Lai, F et al Gene, 248: 117-125 (2000)) were also significantly elevated. Deregulated expression of the above 19 genes may result in increased rate of cell division, mis-segregation of chromosomes, chromosomal instability and aneuploidy that may result in developing cancer.

Increased nucleic acid biosynthesis. To meet the need for nucleic acids for increased cell division, three genes that are involved in the synthesis of pyrimidines, thymidine kinase I (L. J. Bellow Exp. Cell. Res. 89:263-274 (1974)), ribonucleotide reductase M2 (RRM2) (Fan, H et al Proc. Natl. Acad. Sci. 93: 14036-40 (1996)) and thymidylate synthase Gotoh, O, J. Biol. Chem. 265: 20277-20284 (1990)) were increased. One of the enzymes involved in the degradation of purines, adenylate kinase (AK5) (Von Rompay, A. R et al Eur. J. Biochem. 261: 509-514 (1999)) was down regulated.

Increased estrogen levels. It is now very well established that estrogen feeds the breast cancer cells to survive and progress (Gruber, C. J. et al. New E. J. Med. 346: 340-352 (2002)). In ADHC, estrogen levels seem to be higher due to increased synthesis by elevated levels of HSD17B1(17∃-hydroxysteroid dehydrogenase type 1) Gunnarsson, C et al Oncogene, 22:34-40 (2002)) and decreased degradation because of down regulation of UGT2B23, uridine diphosphate-glucuronosyl transferase (Barvier, O et al Endocrinology, 140:5538-5548 (1999)).

Deregulated Cell-Cell and Cell-Extracellular Matrix (ECM) interactions due to elevated expression of Matrix Metalloproteinases (MMPs). Recent research has revealed that MMPs influence both intracellular and extracellular processes that underlie cancer development, namely cell dissociation, death and division (Hojili, C. V et al British J. Cancer, 89:1817-1821 (2002)). Our data show that MMP-1, MMP-3, MMP-9, MMP-11, MMP-12, MMP-13, and MMP-19, plasminogen activator (urokinase receptor, PLAUR), and a lysosomal cystein protease, Cathepsin C, were elevated in ADHC tissues. In addition, two serine/cystein protease inhibitors (SERPINA 3- and 5) which inhibit activities of proteases were down regulated. Elevated levels of these proteases may lead to disassociation of cells from ECM, cleavage of mitogen binding proteins to release growth factors that promote cells division and evade apoptosis that may contribute to carcinogenesis.

Altered cell surface architecture. In addition to Cell-ECM interactions, homotypic and heterotypic intercellular interactions play a very important role in maintenance of cell-cell contact, communication of signals into the cellular interior and interactions with cytoskeletal elements that produce changes in cell motility, migration, proliferation and shape. Alterations in the cell surface molecules cause perturbations in cell polarity and architecture that leads to malignancy (Daxbury, M. S et al Oncogene, 23: 465-73 (2004)). We observed alterations of at least three classes of cell surface molecules, namely carcinoembryonic antigen related cell adhesion molecules (CEACAM5 and CEACAM6), galectins (5 & 9), and five non-conventional proto-cadherins CDH11, DSC2, and VCAM1 and CELSR1-, and -2.

Differentially altered signal transduction processes. We examined specifically genes of growth factor families and kinases. Our data showed that three growth factor binding proteins, latent transforming growth factor beta binding proteins, LTBP3- and 4, and IGFBP2, IGF binding protein 2 were down regulated. In addition, GRB2, growth factor receptor bound protein 2, that forms a complex with EGFR (Matuoka, K et al EMBO J., 12:3467-3473 (1993)) and Ras specific guanine nucleotide exchange factor and mediates EGF and PDGF induced activation of Ras, and EGFL6 (Yeung, G et al Genomics, 62:304-307 (1999)) that is involved in regulation of cell cycle and proliferative responses and expressed in several cancers were increased. Although none of the MAP kinase signaling pathway genes was altered at this stage, two closely related genes MAP4K1 and TOPK, were highly up regulated.

Activated proto-oncogenes and diminished tumor suppressors. At the stage of ADHC, at least four proto-oncogenes that were shown to increase cell proliferation by different pathways were up-regulated. One of increased oncogenes was RET1, (RET proto-oncogene), a cell surface tyrosine kinase, that transduces signals to increase cell proliferation (Iwahashi, N et al Biochem. Biophys. Res. Commun. 294:642-649 (2000)). Another oncogene was NET (neuro-epithelial transforming gene 1), which was known to induce tumors in mice. Although we did not find alterations in WNT expression, a protein that is regulated by it, WISP1 (WNT1 inducible signaling pathway protein 1) (Xu et al Genes Dev. 14:585-595 (2000)), and recognized as an oncogene, was increased. A fourth oncogene that increased significantly was MAFB (V-maf musculoaponeurotic fibro sarcoma onco gene homo log B) (Ni shizawo, M et al Proc. Natl. Acad. Sci. 86: 7711-7715 (1989)), which was shown to play an important role in the regulation of lineage-specific hematopoiesis. In addition, expression of two tumor suppressor genes that down-regulate WNT oncogene, WIF1, a WNT inhibitory factor (Wisssman, C et al J. Pathol. 201:204-212 (2003)), and FRZB, a secreted Frizzled-related protein were down regulated. Both WIF1 and FRZB were shown to be down regulated in breast and other cancers.

Reduced apoptosis and cellular detoxification processes. In ADHC cells, BCL2A1, which was shown to act as an anti-, and pre-apoptotic regulator by inhibiting the release of pro-apoptotic caspase activation components and highly expressed in breast cancer (Werner, A. B et al J. Biol. Chem, 277: 22781-22788 (2002)), was highly increased. In addition, ADHC tissues also have reduced levels of detoxifying enzymes, glutathione-S-transferase and two Cytochromone P450 related genes, CYP4B1 and CYP1A1. Down regulation of these genes could contribute to increased cellular levels of mutagenic free radicals and anti-metabolites respectively. And

High lymphocytic infiltrate. Of the 540 differentially altered gene probes, over 150 up-regulated probes were associated with lymphocytic infiltrate that include several genes expressed primarily by B and T lymphocytes (Supplement, Table 1). This class of genes clearly separated ADHC from ADH. It was previously reported that higher lymphatic infiltrate was associated with aggressive tumors from patients who had BRCA1 mutations and ER∀-negative tumors and the only factor other than ER∀ status that segregates breast tumors into two groups by unsupervised clustering (Van't Veer et al Nature, 415: 530-535 (2002)). Based on the above reports, it is concluded that ADHC are more aggressive precancerous lesions than ADH, an indication they will progress to IBC.

### Example 4

This example demonstrates that one of the highly up-regulated genes in ADHC, MMP-1, is an excellent breast cancer predictive marker. MMP-1 is an enzyme that degrades extracellular matrix. Elevated levels of MMP-1 have been reported in several cancers including breast cancer (Migita T et al, Int. J. Cancer, 84, 74-79 (19999)). Animal studies have suggested that over expression of MMP-1 protein has a role in initiating mammary tumorigenesis by degrading stroma and releasing growth factors and other mitogenes for epithelial cells (Hojilia et al Br. J. Cancer 89, 1817-1821 (2003) Duffy, M. J. et al, Breast Cancer Res, 2, 252-257 (2000)). MMP-1 was shown to specifically degrade insulin-like growth factor (IGF) binding proteins 2, 3 and 5, fibroblast growth factor (FGF) binding protein and transforming growth factor (TGF)-beta binding protein and release IGF, FGF and TGF-beta (Vu, T et al, Genes Dev 14, 2123-2133 (2000)). MMP-1 was chosen for testing retrospectively for prediction of breast cancer development, because it was one of the most highly upregulated markers (ratio=35.5, p=6.5E-08) in ADHC and 2) its expression was at undetectable levels in ADH samples. The expression of MMP-1 was retrospectively validated by immunohistochemistry in a total of 108 formalin fixed paraffin embedded archival tissues. Among the 108 tissues, 44 were ADH (atypical ductal hyperplastic tissues from patients who had no previous history of cancer and did not develop cancer in 5 years after the diagnosis), 44 ADHC (ADH tissues from patients who subsequently developed cancer in 5 years or had both cancer and ADH concurrently) and 20 non-ADH benign tissues from patients who subsequently developed cancer. The histology of non-ADH benign tissues ranged from fibrocystic change to papilloma (please see Table 4). All non-ADH tissues are characterized by hyperproliferation with various morphological characteristics. These are also called as Usual Ductal Hyperplasias (UDH). Five tissues from each that were diagnosed as DCIS and IBC as positive controls and normal breast tissues from women who underwent reduction mammoplasty for cosmetic reasons were also included as negative controls. By immunohistochemical analysis, all DCIS and IBC tissues were strongly positive for MMP-1. Of the 44 ADH (controls), 7 showed some positivity for MMP-1. Of the 44 ADHC tissues, 7 showed the absence of MMP-1 and the rest were positive. Among the 20 non-ADH benign tissues from patients who subsequently developed cancer, all were positive for the presence of MMP-1 (Table 4). Representative micrographs from normal, non-ADH benign (hyperplasia and fibrocystic change), ADH, ADHC, DCIS and IBC are presented in FIG. 7. MMP-1 staining was seen both in stroma and ductal epithelial cells in all the positive tissues (Poola et al Nature Medicine, 11: 481-483 (2005)). These results together with real-time PCR data strongly demonstrated that MMP-1 expression was associated with development of cancer irrespective of the morphological appearance of precancerous tissues. These results demonstrate that MMP-1 expression in precancerous lesions is strongly predictive of cancer development.

### Example 5

This example demonstrates that another one of the highly up-regulated genes in ADHC, CEACAM6, is also an excellent breast cancer predictive marker. It was also one of the most highly up-regulated markers (ratio=37 and p value=9.5E-06) in ADHC and its expression was at undetectable levels in ADH samples (GEO data bases, Accession # GSE2429). CEACAM6 is a glycosylphosphatidylinositol—anchored cell surface protein that functions as a homotypic intercellular adhesion molecule and can block anoikis (apoptotic response induced in normal cells by inadequate or inappropriate adhesion to substrate) of several different cell types. Elevated levels of CEACAM6 was shown to play an instrumental role in tumorigenesis by disrupting the functions of integrins which in turn affect cell-ECM interactions, cell polarity and architecture and inhibition of cell differentiation (Stanners, et al Basic and Clinical Perspective 5, 57-79 (1998)) and Ilantzis, C. et al Neoplasia, 4, 151-63 (2002)). It is over-expressed in a number of human malignancies including breast cancers (Jantscheff, P. et al, J. Clinical Oncology 21, 3638-46 (2003). Duxbury, M. S. et al Oncogene. 23, 465-473 (2004), and Lacroix, M. et al The International J. Biological Markers. 17, 5-23 (2002)) and increased levels of CEACAM6 is inversely correlated to differentiation state of cancer cells. CEACAM6 was extensively investigated in gastrointestinal cancers. Silencing of CEACAM6 gene impairs anoikis and in vivo metastatic ability of pancreatic adnocarcinoma cells (Duxbury, M. S. et al Oncogene. 23, 465-473 (2004)). It was reported to be up-regulated at the early stages of colorectal cancers such as early adenomas and hyperplastic polyps (Scholzel, S., et al. American J. Pathology. 156, 595-605 (2000)). Increased level of CEACAM6 was also shown to be an independent prognostic factor in colorectal cancers (Ilantzis, C. et al Neoplasia, 4, 151-63 (2002) and Jantscheff, P. et al, J. Clinical Oncology 21, 3638-46 (2003)).

In addition to MMP-1, CEACAM6 was established as a predictive marker for the following reasons: Because of the complexity of breast carcinogenesis and heterogeneity of precancerous lesions more than one molecular marker would be needed for making definitive predictions of subsequent cancer development. Screening and prediction of subsequent development of cancer based on multiple molecular markers both individually and in combination will be more reliable and will have higher patient acceptance in a clinical situation. The Sensitivity will also be higher when multiple markers will be considered together than a single marker. In addition, establishing multiple marker expression in precancerous tissues that are highly likely to progress to cancer could lead to design of novel targeted prophylactic molecular therapies for treating pre-malignant lesions and preventing from IBC development. CEACAM6 expression in precancerous breast tissues was studied to validate it as a predictive marker for breast cancer development both individually and in combination with MMP-1 so that predictions could be made based on at least two markers in a clinical situation for patient acceptability and reliability. Establishing CEACAM6 in patients with precancerous tissues could also have therapeutic implications. A number of recent studies have established that blocking cell-cell adhesion with CEACAM6 targeted antibodies are excellent blockers of cancer progression (Blumenthal R D et al Cancer Res. 65, 8809-8817 (2005); Dexbury M S et al. Biochem. Biophys. Res. Commun. 317, 837-843, 2004; Chan C H et al Mol. Therapy, 9, 775-785, 2004; Chester K A. et al Cancer Chemother. pharm. 46, 2000; Xu X. Cancer Res. 60, 4475-4484, 2000; Soeth E. et al. Clinical Cancer Res. 7, 2022-2030, 2001; and Wirth T et al. Clin. Exp. Metastasis. 19, 155-160, 2002;) and vaccines based on CEACAM6 in clinical trials for preventing the progression of breast as well as colon cancers have been highly promising (Marshall J. Semin. Oncol. 30, 30-36, 2003; Marshall J L et al. J. Clin. Oncol. 23, 659-61, 2005; and Moses M A, et al Clin. Cancer Res. 11, 3017-24, 2005). Thus establishing the expression of CEACAM6 in precancerous tissues that are highly likely to develop into cancer could become the basis for treating the patients with CEACAM6 targeted therapies and/or vaccinating them to prevent from developing cancer.

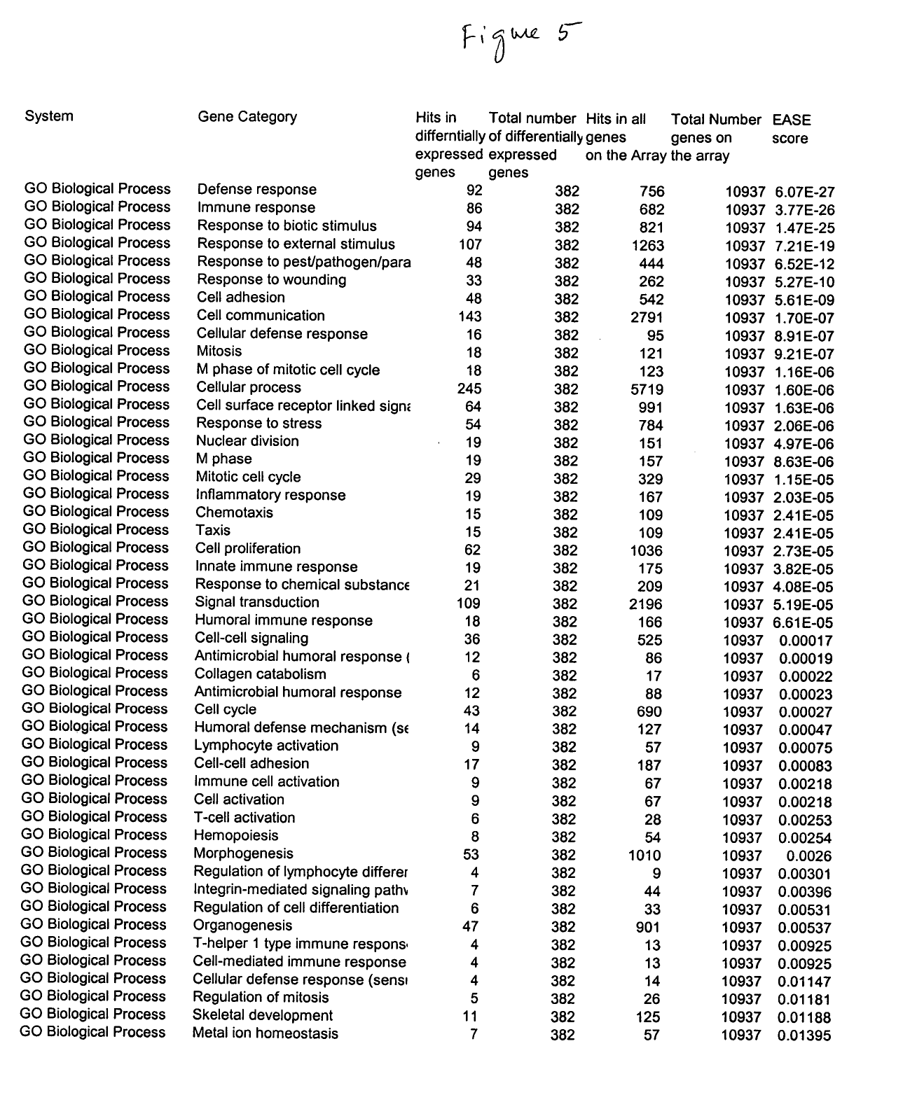

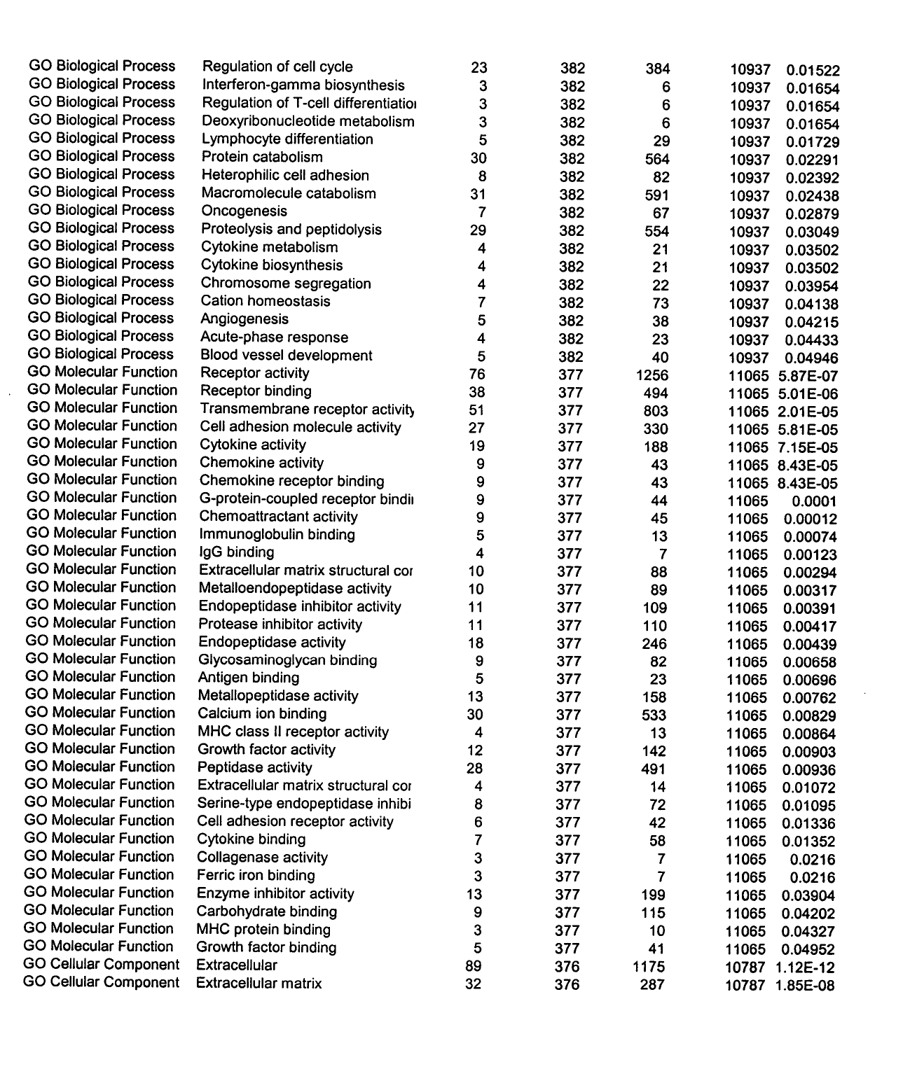

The expression of CEACAM6 was retrospectively validated by immunohistochemistry in a total of 108 formalin fixed paraffin embedded archival tissues. Among the 108 tissues, 44 were ADH (atypical ductal hyperplastic tissues from patients who had no previous history of cancer and did not develop cancer in 5 years after the diagnosis), 44 ADHC (ADH tissues from patients who subsequently developed cancer in 5 years or had both cancer and ADH concurrently) and 20 non-ADH benign tissues from patients who subsequently developed cancer. Five tissues that were diagnosed as IBC were included as positive controls and normal breast tissues from women who underwent reduction mammoplasty for cosmetic reasons were also included as negative controls. By immunohistochemical analysis, all IBC tissues were strongly positive for CEACAM6. Of the 44 ADH (controls), 9 showed some positivity for CEACAM6. Of the 20 non-ADH benign tissues from patients who subsequently developed cancer, 11 were negative and 9 were positive. Of the 44 ADHC tissues, 4 were negative and 40 were positive (Table 4). Representative micrographs from normal, non-ADH benign (fibrocystic change), ADH, ADHC, and IBC are presented in FIG. 8. CEACAM6 staining was seen both in cytoplasm and cell membrane in all the positive tissues. These results demonstrate that CEACAM6 expression in precancerous lesions is strongly predictive of cancer development (Poola et al (2006) Clinical Cancer Res. 12, 4773-4873).

TABLE 4
MMP-1 and CEACAM6 PROTEIN EXPRESSION IN ADH, ADHC AND NON-ADH BENIGN TISSUES BY MMUNOHISTOCHEMISTRY

| Histological | Cancer Development | | | | | | | | | |
|---|---|---|---|---|---|---|---|---|---|---|
| Type of Pre- | (Years pre-/post | Pre- | | Age at | | | | | * | |
| Cancerous | diagnosis of | cancerous | Cancer | which cancer | Histological | Grade of | ER/PR | Nodal | MMP1 | * |
| Lesion | Precancerous lesion) | Breast | Breast | developed | type of cancer | cancer | status | status | in stroma | CEACAM6 |
| ADH | 7 Free | NA | NA | NA | NA | NA | NA | NA | 0 | 0 |
| ADH | 7 Free | NA | NA | NA | NA | NA | NA | NA | 0 | 0 |
| ADH | 7 Free | NA | NA | NA | NA | NA | NA | NA | 0 | 0 |
| ADH | 7 Free | NA | NA | NA | NA | NA | NA | NA | 0 | 0 |
| ADH | 7 Free | NA | NA | NA | NA | NA | NA | NA | 0 | 3 |
| ADH | 7 Free | NA | NA | NA | NA | NA | NA | NA | 0 | 0 |
| ADH | 7 Free | NA | NA | NA | NA | NA | NA | NA | 0 | 0 |
| ADH | 7 Free | NA | NA | NA | NA | NA | NA | NA | 1 | 0 |
| ADH | 8 Free | NA | NA | NA | NA | NA | NA | NA | 0 | 0 |
| ADH | 7 Free | NA | NA | NA | NA | NA | NA | NA | 0 | 0 |
| ADH | 8 Free | NA | NA | NA | NA | NA | NA | NA | 0 | 0 |
| ADH | 5 Free | NA | NA | NA | NA | NA | NA | NA | 0 | 1 |
| ADH | 3 Free | NA | NA | NA | NA | NA | NA | NA | 0 | 0 |
| ADH | 7 Free | NA | NA | NA | NA | NA | NA | NA | 1 | 0 |
| ADH | 3 Free | NA | NA | NA | NA | NA | NA | NA | 0 | 0 |
| ADH | 3 Free | NA | NA | NA | NA | NA | NA | NA | 0 | 0 |
| ADH | 3 Free | NA | NA | NA | NA | NA | NA | NA | 0 | 0 |
| ADH | 7 Free | NA | NA | NA | NA | NA | NA | NA | 1 | 0 |
| ADH | 5 Free | NA | NA | NA | NA | NA | NA | NA | 0 | 0 |
| ADH | 5 Free | NA | NA | NA | NA | NA | NA | NA | 0 | 0 |
| ADH | 5 Free | NA | NA | NA | NA | NA | NA | NA | 0 | 0.5 |
| ADH | 5 Free | NA | NA | NA | NA | NA | NA | NA | 0 | 1 |
| ADH | 5 Free | NA | NA | NA | NA | NA | NA | NA | 0 | 0 |
| ADH | 7 Free | NA | NA | NA | NA | NA | NA | NA | 1 | 0 |
| ADH | 5 Free | NA | NA | NA | NA | NA | NA | NA | 0 | 0 |
| ADH | 5 Free | NA | NA | NA | NA | NA | NA | NA | 0 | 0 |
| ADH | 5 Free | NA | NA | NA | NA | NA | NA | NA | 0 | 0 |
| ADH | 6 Free | NA | NA | NA | NA | NA | NA | NA | 0 | 0.5 |
| ADH | 5 Free | NA | NA | NA | NA | NA | NA | NA | 0 | 0 |
| ADH | 9 Free | NA | NA | NA | NA | NA | NA | NA | 0 | 0 |
| ADH | 8 Free | NA | NA | NA | NA | NA | NA | NA | 1 | 2 |
| ADH | 7 Free | NA | NA | NA | NA | NA | NA | NA | 0 | 0 |
| ADH | 7 Free | NA | NA | NA | NA | NA | NA | NA | 0 | 0 |
| ADH | 7 Free | NA | NA | NA | NA | NA | NA | NA | 0.5 | 1 |
| ADH | 7 Free | NA | NA | NA | NA | NA | NA | NA | 0 | 0 |
| ADH | 7 Free | NA | NA | NA | NA | NA | NA | NA | 0 | 0 |
| ADH | 7 Free | NA | NA | NA | NA | NA | NA | NA | 0 | 0 |
| ADH | 6 Free | NA | NA | NA | NA | NA | NA | NA | 0 | 0 |
| ADH | 6 Free | NA | NA | NA | NA | NA | NA | NA | 0 | 0 |
| ADH | 6 Free | NA | NA | NA | NA | NA | NA | NA | 0 | 0 |
| ADH | 6 Free | NA | NA | NA | NA | NA | NA | NA | 0.5 | 0 |
| ADH | 6 Free | NA | NA | NA | NA | NA | NA | NA | 0 | 0 |
| ADH | 6 Free | NA | NA | NA | NA | NA | NA | NA | 0 | 0.5 |
| ADH | 6 Free | NA | NA | NA | NA | NA | NA | NA | 0 | 1 |
| Fibrocystic | 1 Pre | Left | Left | 52 | Intraductal | II | ND | − | 2 | 0 |
| Change | | | | | carcinoma | | | | | |
| Stromal | 1 Pre | Right | Right | 72 | Invasive ductal | II | ND | − | 2 | 0 |
| Fibrosis | | | | | carcinoma | | | | | |
| Fibrocystic | 7 Pre | Left | Left | 59 | Metastatic | III | ND | + | 1 | 0 |
| Change | | | | | adenocarcinoma | | | | | |
| Lobular | 3 Pre | Left | Left | 41 | Lobular | ND | ND | − | 2 | 0 |
| Hyperplasia | | | | | carcinoma insitu | | | | | |
| Intraductal | 3 Pre | Right | Right | 71 | Invasive tubular | I | −/− | − | 2 | 0 |
| Hyperplasia | | | | | ductal carcinoma | | | | | |
| Intraductal | 4 Pre | Right | Right | 61 | Invasive ductal | III | ND | − | 2 | 1 |
| Papilloma | | | | | and lobular | | | | | |
| | | | | | carcinoma | | | | | |
| Fibrocystic | 2 Pre | Left | Left | 80 | Invasive ductal | III | +/− | − | 1 | 2 |
| Change | | | | | carcinoma | | | | | |
| Stromal | 3 Pre | Right | Right | 64 | Infiltrating ductal | II | +/+ | − | 2 | 1 |
| Fibrosis | | | | | carcinoma | | | | | |
| Intraductal | 3 Pre | Left | Left | 62 | Invasive ductal | III | +/− | + | 2 | 1 |
| Papilloma | | | | | carcinoma | | | | | |
| Epithelial | 2 Pre | Left | Left | 70 | Lobular cancer | ND | ND | − | 2 | 0.5 |
| Hyperplasia | | | | | in situ | | | | | |
| Hyperplasia, | 5 Pre | Right | Right | 71 | Invasive ductal | II | ND | − | 2 | 0 |
| Fibrocystic | | | | | carcinoma | | | | | |
| Change | | | | | | | | | | |
| Fibrocystic | 4 Pre | Left | Left | 76 | Invasive ductal | III | −/− | + | 2 | 1 |
| Change | | | | | carcinoma | | | | | |
| Fibrocystic | 9 Pre | Left | Left | 70 | Ductal | III | −/− | − | 3 | 0 |
| Change | | | | | carcinoma in situ | | | | | |
| Fibrocystic | 5 Pre | Right | Right | 34 | Invasive ductal | III | −/− | + | 2 | 0 |
| Change | | | | | carcinoma | | | | | |
| Intraductal | 5 Pre | Right | Right | 74 | Invasive | II | −/− | + | 2 | 1 |
| Hyperplasia | | | | | adeosquamous | | | | | |
| | | | | | carcinoma | | | | | |
| Intraductal | 5 Pre | Left | Left | 48 | Invasive | I | +/− | − | 3 | 0 |
| Papilloma | | | | | papillary | | | | | |
| | | | | | carcinoma | | | | | |
| Fibrocystic | 1 Pre | Right | Right | 35 | Invasive ductal | III | ND | + | 2 | 0 |
| Change | | | | | carcinoma | | | | | |
| Intraductal | 5 Pre | Left | Left | 48 | Invasive | I | ND | ND | 1 | 1 |
| Papilloma | | | | | papillary | | | | | |
| | | | | | carcinoma | | | | | |
| Intraductal | 3 Pre | Left | Left | 62 | Diffuse | ND | ND | ND | 0.5 | 0 |
| Papilloma | | | | | infiltrating | | | | | |
| | | | | | lobular | | | | | |
| | | | | | carcinoma | | | | | |
| Fibrocystic | 3 Pre | Right | Right | 66 | Invasive ductal | II | ND | + | 1 | 1 |
| Changes | | | | | carcinoma | | | | | |
| ADHC | 2 Pre | Right | Right | 70 | Infiltrating ductal | II | +/− | + | 3 | 4 |
| | | | | | carcinoma | | | | | |
| ADHC | 1 Pre | Right | Right | 69 | Invasive ductal | III | ND | − | 3 | 4 |
| | | | | | carcinoma | | | | | |
| ADHC | 2 Pre | Left | Left | 51 | Intraductal | ND | +/− | − | 2 | 2 |
| | | | | | carcinoma in situ | | | | | |
| ADHC | 7 Pre | Left | Right | 79 | Invasive ductal | III | −/− | + | 3 | 0.5 |
| | | | | | carcinoma | | | | | |
| ADHC | 3 Pre | Left | Left | 70 | Lobular | ND | ND | − | 0 | 3 |
| | | | | | carcioma in situ | | | | | |
| ADHC | 3 Pre | Left | Left | 31 | Invasive ductal | III | ND | − | 2 | 2 |
| | | | | | carcinoma | | | | | |
| ADHC | 1 Pre | Left | Left | 63 | Intraductal | ND | ND | − | 2 | 2 |
| | | | | | carcinoma in situ | | | | | |
| ADHC | 4 Pre | Left | Left | 70 | Intraductal | ND | ND | − | 3 | 2 |
| | | | | | carcinoma in situ | | | | | |
| ADHC | 5 Pre | Right | Left | 41 | Lobular | ND | ND | − | 0 | 2 |
| | | | | | carcioma in situ | | | | | |
| ADHC | 3 Pre | Right | Right | 45 | Invasive ductal | ND | ND | − | 3 | 1 |
| | | | | | carcinoma | | | | | |
| ADHC | 2 Pre | Right | Left | 49 | Infiltrating ductal | ND | ND | − | 3 | 0 |
| | | | | | carcinoma | | | | | |
| ADHC | 5 Pre | Right | Right | 61 | Invasive ductal | ND | ND | − | 3 | 2 |
| | | | | | carcinoma | | | | | |
| ADHC | 2 Pre | Right | Right | 64 | Infiltrating ductal | ND | ND | − | 2 | 1 |
| | | | | | carcinoma | | | | | |
| ADHC | 4 Pre | Right | Left | 49 | Invasive ductal | III | −/− | + | 3 | 2 |
| | | | | | carcinoma | | | | | |
| ADHC | Simultaneous | Same | Same | 80 | Infilterating | III | −/− | − | 4 | 1 |
| | | | | | ductal carcinoma | | | | | |
| ADHC | Simultaneous | Same | Same | 48 | Intraductal | II | ND | − | 4 | 1 |
| | | | | | carcinoma | | | | | |
| ADHC | Simultaneous | Same | Same | 45 | Invasive dutcal | III | ND | − | 3 | 0 |
| | | | | | carcinoma | | | | | |
| ADHC | Simultaneous | Same | Same | 62 | Insitu lobular | II | ND | − | 3 | 2 |
| | | | | | carcinoma | | | | | |
| ADHC | Simultaneous | Same | Same | 61 | Insitu ductal | II | ND | − | 2 | 1 |
| | | | | | carcinoma | | | | | |
| ADHC | Simultaneous | Same | Same | 55 | Invasive ductal | III | −/− | + | 4 | 2 |
| | | | | | carcinoma | | | | | |
| ADHC | Simultaneous | Same | Same | 40 | Insitu lobular | ND | ND | − | 3 | 3 |
| | | | | | carcinoma | | | | | |
| ADHC | Simultaneous | Same | Same | 61 | Invasive lobular | III | ND | − | 3 | 1 |
| | | | | | carcinoma | | | | | |
| ADHC | Simultaneous | Same | Same | 70 | Ductal | ND | ND | − | 4 | 2 |
| | | | | | carcinoma insitu | | | | | |
| ADHC | Simultaneous | Same | Same | 37 | Insitu ductal | III | ND | − | 3 | 1 |
| | | | | | carcinoma | | | | | |
| ADHC | Simultaneous | Same | Same | 47 | Invasive lobular | ND | +/− | − | 4 | 3 |
| | | | | | carcinoma | | | | | |
| ADHC | Simultaneous | Same | Same | 50 | Invasive ductal | II | +/− | + | 0 | 1 |
| | | | | | carcinoma | | | | | |
| ADHC | Simultaneous | Same | Same | 86 | Invasive Ductal | III | +/+ | + | 3 | 0 |
| | | | | | carcinoma | | | | | |
| ADHC | Simultaneous | Same | Same | 55 | Ductal | ND | ND | − | 2 | 1 |
| | | | | | carcinoma insitu | | | | | |
| ADHC | Simultaneous | Same | Same | 70 | Infiltrating ductal | II | +/− | + | 0 | 0 |
| | | | | | carcinoma | | | | | |
| ADHC | Simultaneous | Same | Same | 47 | Ductal | ND | ND | − | 4 | 2 |
| | | | | | carcinoma insitu | | | | | |
| ADHC | Simultaneous | Same | Same | 42 | Insitu Ductal | III | ND | − | 0 | 1 |
| | | | | | carcinoma | | | | | |
| ADHC | Simultaneous | Same | Same | 45 | Insitu ductal | II | +/+ | − | 0 | 2 |
| | | | | | carcinoma | | | | | |
| ADHC | Simultaneous | Same | Same | 71 | Invasive ductal | I | +/+ | + | 4 | 4 |
| | | | | | carcinoma | | | | | |
| ADHC | Simultaneous | Same | Same | 51 | Ductal | II | ND | − | 0 | 1 |
| | | | | | carcinoma insitu | | | | | |
| ADHC | Simultaneous | Same | Same | 48 | Invasive ductal | II | +/+ | − | 4 | 3 |
| | | | | | carcinoma | | | | | |
| ADHC | Simultaneous | Same | Same | 46 | Infiltrating ductal | I | ND | − | 4 | 2 |
| | | | | | carcinoma | | | | | |
| ADHC | Simultaneous | Same | Same | 54 | Invasive ductal | II | +/+ | − | 4 | 3 |
| | | | | | carcinoma | | | | | |
| ADHC | Simultaneous | Same | Same | 59 | Invasive ductal | ND | −/− | − | 3 | 2 |
| | | | | | carcinoma | | | | | |
| ADHC | Simultaneous | Same | Same | 51 | Invasive ductal | II | +/+ | − | 4 | 2.5 |
| | | | | | carcinoma | | | | | |
| ADHC | Simultaneous | Same | Same | 50 | Lobular | ND | +/+ | − | 2 | 1 |
| | | | | | carcinoma insitu | | | | | |
| ADHC | Simultaneous | Same | Same | 55 | Ductal | II | ND | − | 3 | 2 |
| | | | | | carcinoma insitu | | | | | |
| ADHC | Simultaneous | Same | Same | 75 | Medullary ductal | III | +/+ | − | 3 | 1 |
| | | | | | carcinoma | | | | | |
| ADHC | Simultaneous | Same | Same | 52 | Invasive ductal | III | +/+ | − | 4 | 2.5 |
| | | | | | carcinoma | | | | | |
| ADHC | Simultaneous | Same | Same | 69 | Intraductal | ND | ND | − | 3 | 3 |
| | | | | | carcinoma | | | | | |

ND, Not Determined;
NA, Not Applicable,
* The intensity of staining was graded in comparison with an arbitrary value of 5.0 assigned for Invasive Breast Cancer tissues.

### Example

This example demonstrates the Sensitivity (percentage of test precancerous samples that were positive for the marker), Specificity (percentage of control precancerous samples that were negative for the marker), PPV (correctly predicting cancer development in patients who were positive for the marker) and NPV (correctly predicting non-development of cancer in patients who were negative for the marker) of expression of MMP-1 and CEACAM6 markers individually and in combination in precancerous tissues, and p values for association of their expression with cancer development individually and in combination; To determine the specificity, sensitivity, ppv and npv values, the data in Table 4 were analyzed for ROC (Receiver Operating Characteristics) statistics using S-PLUS software. The p values were determined using chi-squared test. The results are shown in Table 5. The results presented in Table 5 demonstrate that both markers are highly predictive of developing cancer, independently, as shown by the p values and ppv values. The predictive power is increased when both markers are combined. Both markers have high sensitivity as individual markers. The sensitivity is increased when both markers are combined together compared to individual markers. The specificity is also very high for individual markers. When both markers are combined the high specificity is maintained although slightly lower than individual markers presumably due to individual variation in expression of these two markers (Table 4). To determine the best way to combine the two markers, we first analyzed the data in Table 4 by logistic regression analyses and obtained the coefficients 1.8 and 1.6 for MMP-1 and CEACAM6 respectively. Based on these, we combined the markers by two ways: 1) because the coefficients are almost the same, the first way we combined the markers was by simply adding the grading scores, and 2) the second way we combined the markers was by first multiplying the marker grading scores with their respective coefficients and then adding those values. We compared the results of the two combinations by using their corresponding ROC curves and found to have no differences.

TABLE 5
ROC Statistics, and p values for MMP-1, CEACAM6 and MMP-1
CEACAM6 in ADH, ADHC and non-ADH Benign Tissues

| Pre- | Sensitivity | | | Specificity | | | PPV | | |
|---|---|---|---|---|---|---|---|---|---|
| cancerous | | | MMP-1 Plus | | | MMP-1 Plus | | | MMP-1 Plus |
| Tissues | MMP-1 | CEA-CAM6 | CEA-CAM6 | MMP-1 | CEA-CAM6 | CEA-CAM6 | MMP-1 | CEA-CAM6 | CEA-CAM6 |
| Non-ADH | 0.88 | 0.73 | 0.97 | 0.89 | 0.86 | 0.77 | 0.92 | 0.89 | 0.86 |
| benign, ADH | | | | | | | | | |
| & ADHC | | | | | | | | | |

| | | |
|---|---|---|
| Pre- | NPV | P values |

| cancerous | | | MMP-1 Plus | | | MMP-1 Plus |
|---|---|---|---|---|---|---|
| Tissues | MMP-1 | CEA-CAM6 | CEA-CAM6 | MMP-1 | CEA-CAM6 | CEA-CAM6 |
| Non-ADH | 0.83 | 0.69 | 0.94 | 2 × 10⁻¹⁴ | 3 × 10⁻⁹ | 5 × 10⁻¹⁵ |
| benign, ADH | | | | | | |
| & ADHC | | | | | | |

* All the calculations were done using the expression levels (grading scores) at 0.5-1.0 for both markers.

### Example

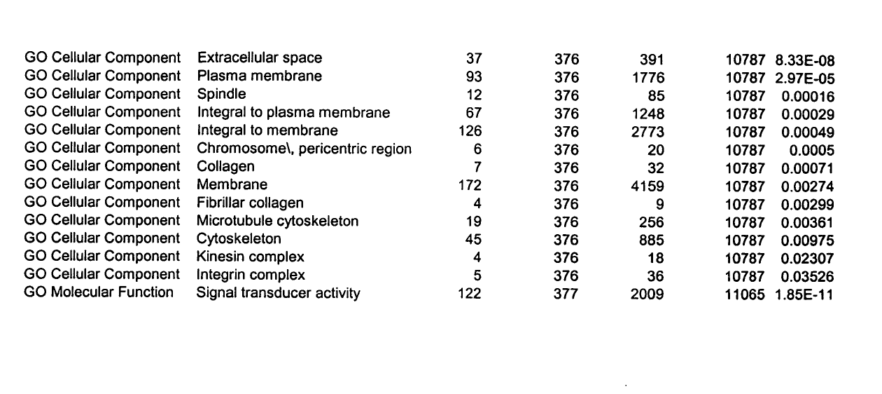

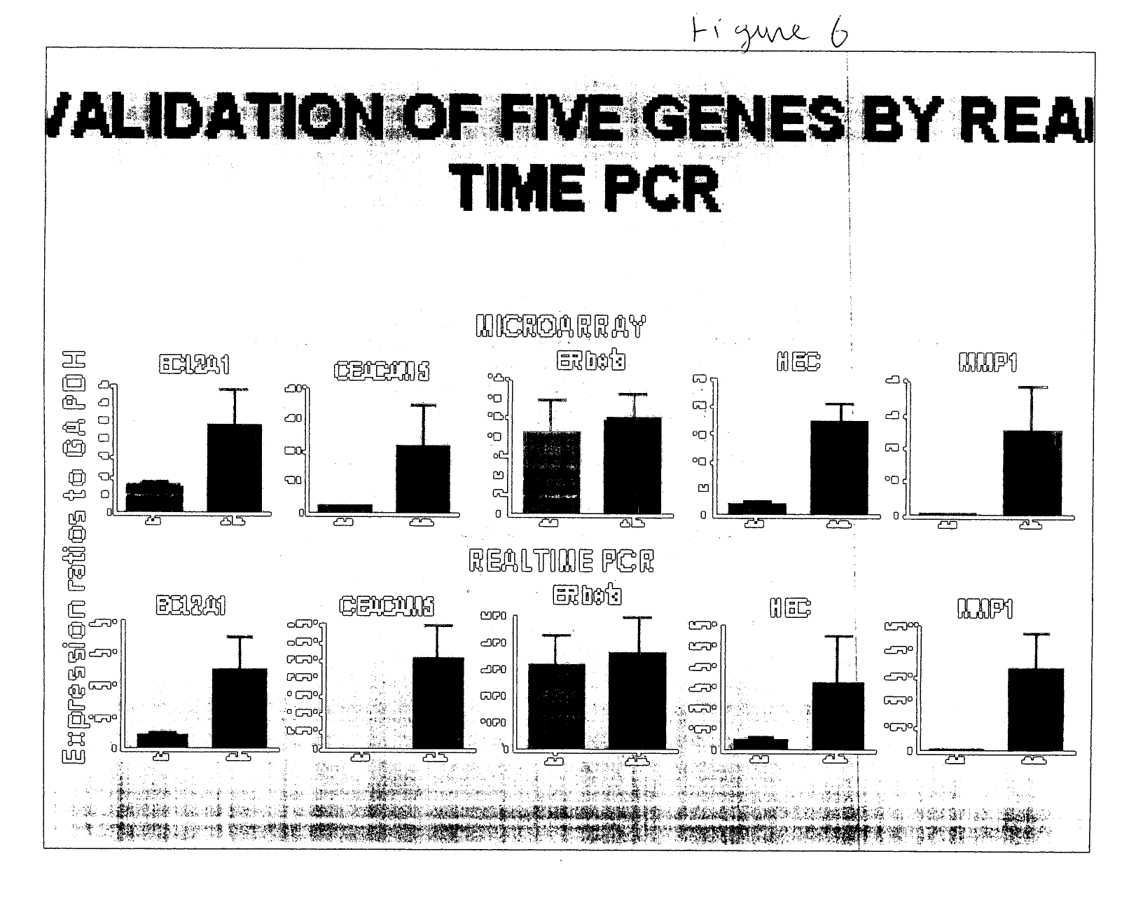

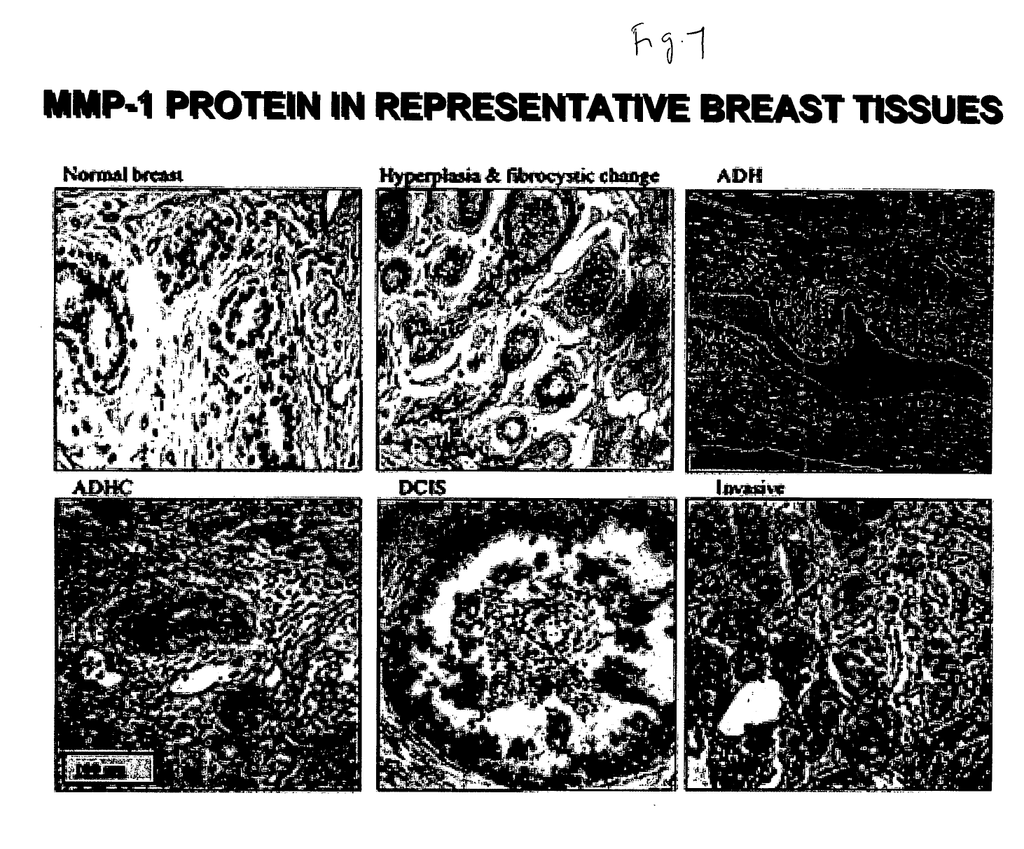

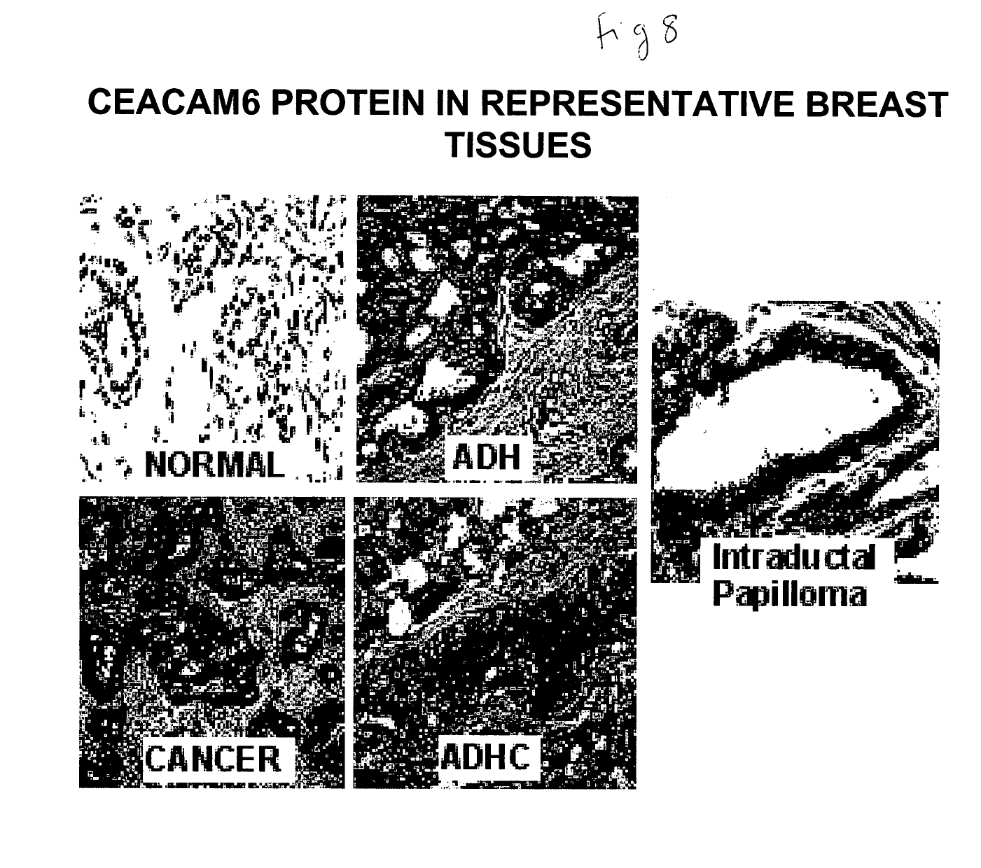

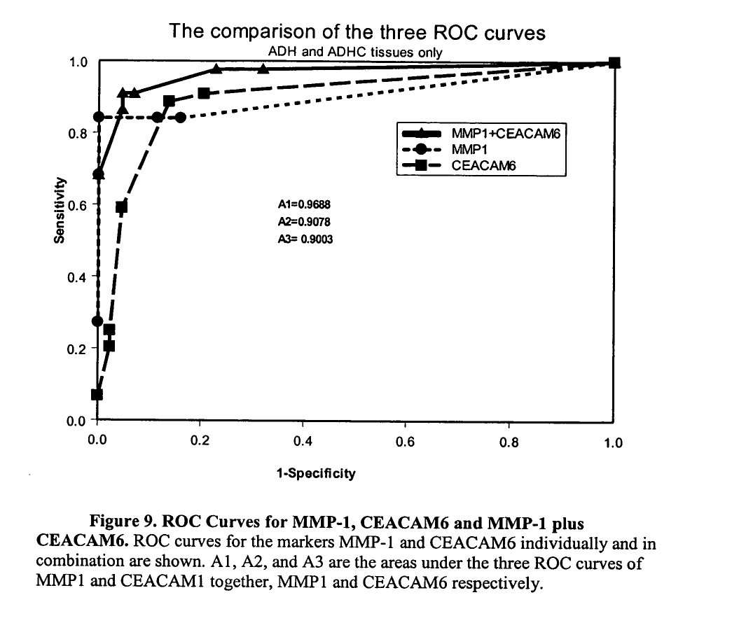

This example demonstrates the ROC curves for MMP-1, CEACAM6 and MMP-1 and CEACAM6 combined. The ROC curves were drawn with the expression data by immunohistochemistry (Table 4) using S-PLUS software. The ROC curves were generated as follows. For each threshold value, if the measured value (marker grading score, or sum of two grading scores for the two markers combined in our situations) is greater or equal to the threshold value, then it is considered as a positive test, otherwise it is a negative test. Thus, each threshold value determines a point with coordinates (1-specificity, sensitivity). The ROC curves were generated for individual markers and in combination (combined by simply adding the grading scores (first way above)). For MMP-1 and CEACAM6 separately, the threshold values were the five grading scores (0.5, 1, 2, 3 and 4). For MMP-1 and CEACAM6 combined, the threshold values were 0.5, 1, 1.5, 2, 2.5, 3, and 3.5 which give 100% specificity. All the ROC curves were generated by connecting all the points determined by all the threshold values in an increasing order. The ROC curves are shown in FIG. 9. As seen in FIG. 9, ROC curves for both markers show very high sensitivity and specificity for individual markers. The sensitivity is considerably increased when both markers are taken together and the sensitivity has only slightly decreased presumably due to variation in the expression of these markers in individual samples.

### Example 5

This example demonstrates the feasibility of detection of MMP-1 in ductal lavage samples by conventional PCR. Detection of MMP-1 in ductal lavage samples is demonstrated so that it could be used as a marker to detect precancerous cells in the ductal cells obtained by ductal lavage collection procedure before mammographicaly detectable lesions are formed and predict cancer development. MMP-1 expression was first assayed by conventional PCR using MMP-1 specific sense and anti-sense primers, 5′AGATCATCGGGACAACTCTCCTT3′ (Position, bp 568-591) and 5′TAAGCAGCTTCAAGCCCATT3′ (Position, bp 1047-1066) (Pubmed Accession No. NM_{—}002421) respectively in a total of 10 microliters. All the 10 micro-liters of the PCR reaction volume was analyzed by 1% agarose gel electrophoresis to test for the presence of MMP-1 PCR product. ADHC and Invasive breast cancer tissues were used as positive tissues and ADH as negative controls. The data presented in FIG. 10 demonstrate that it is feasible to detect MMP-1 mRNA in ductal lavage by conventional RT PCR. The expected MMP-1 product (which was confirmed by cloning and sequence analysis) could be seen in three ductal lavage samples in FIG. 10. The top panel in the figure demonstrate the positive (ADHC and Invasive Breast Cancer) and negative (ADH) tissue controls.

### Example 6

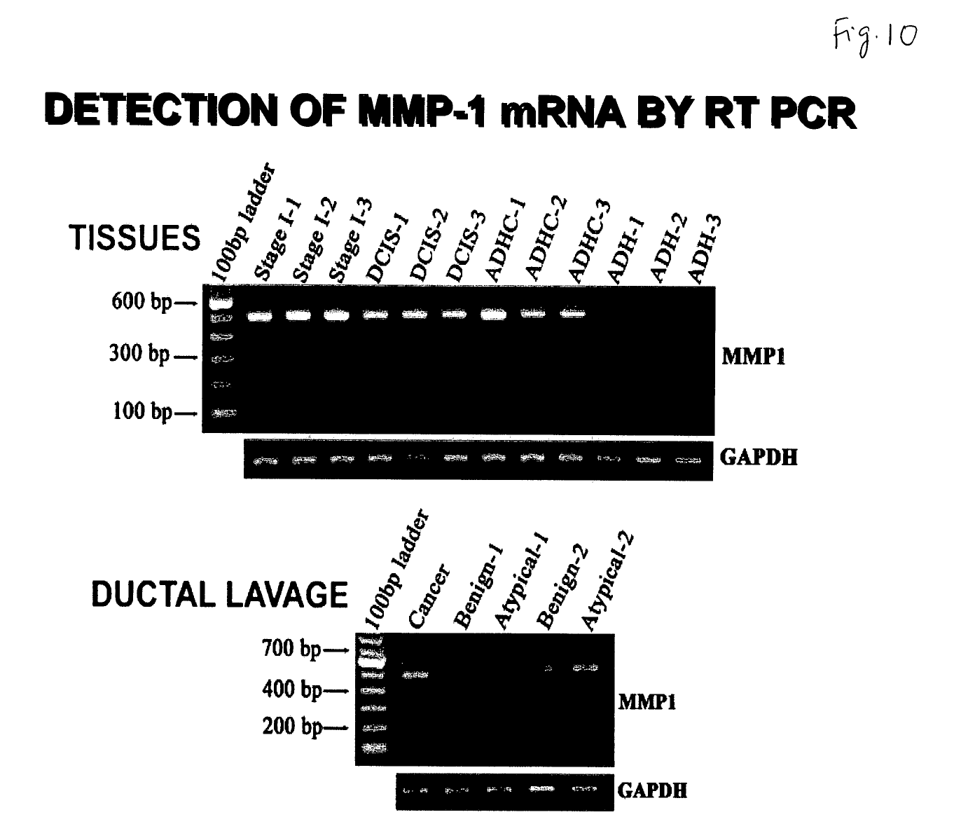

This example demonstrates that morphologically diverse types of ductal cells could express the predictive marker, MMP-1. Out of 49 ductal lavage samples we studied (Table 6) for the presence of MMP-1, at least three samples that were cytologically diagnosed as atypia were negative for the presence of MMP-1 and two samples which were cytologically diagnosed as beign were positive for MMP-1. An example from each is shown in FIG. 11. These results provide strong support for a hypothesis that cells diagnosed as atypia by cytology could be different in molecular marker composition and morphologically benign appearing cells could have cancer promoting molecular markers. Thus MMP-1 expression in cells obtained from procedures such as ductal lavage collection could potentially be used to predict breast cancer development instead of relying only on the morphological shape of the ductal cells.

TABLE 6
MMP-1 Expression in ductal lavage
by Quantitative real time PCR

| | | Nipple | MMP-1 |
|---|---|---|---|
| No | Cytological Diagnosis | Discharge | Presence |
| 1 | Benign with multinucleated histiocytic | No | Positive |
| | giant cells and rare mixed | | |
| | inflammatory cells | | |
| 2 | Benign, mixed inflammatory cells | Yes | Negative |
| 3 | Cancer | Yes | Positive |
| 4 | Benign, fragments of hyperplastic | Yes | Negative |
| | duct-lining cells | | |
| 5 | Benign | No | Negative |
| 6 | Fibrocystic disease with apocrine | Yes | Negative |
| | metaplasia | | |
| 7 | Benign, honeycomb appearing nests | Yes | Negative |
| | of hyperplastic ductal cells | | |
| 8 | Benign | Yes | Negative |
| 9 | Benign | Yes | Negative |
| 10 | Benign, foam cells and mixed | Yes | Negative |
| | inflammatory cells | | |
| 11 | Atypical epithelial cells | Yes | Positive |
| 12 | Hyperplasia to mild atypia | Yes | Negative |
| 13 | Benign, foam cells and inflammatory | No | Negative |
| | cells | | |
| 14 | Benign, foam cells and inflammatory | Yes | Negative |
| | cells | | |
| 15 | Benign, foam cells and mixed | Yes | Negative |
| | inflammatory cells | | |
| 16 | Hyperplasia with atypia | Yes | Positive |
| 17 | Benign, foam cells and mixed | Yes | Negative |
| | inflammatory cells | | |
| 18 | Hyperplasia with mild atypia, foam | Yes | Positive |
| | cells & mixed inflammatory cells | | |
| 19 | Hyperplasia, foam cells and mixed | Yes | Negative |
| | inflammatory cells | | |
| 20 | Benign, foam cells and mixed | Yes | Positive |
| | inflammatory cells | | |
| 21 | Hyperplasia to atypia, foam ductal/ | Yes | Positive |
| | histiocytic cells | | |
| 22 | Hyperplasia to atypia | Yes | Positive |
| 23 | Benign, foam cells | No | Negative |
| 24 | Papillary adenoma, hyperplasia, atypia | Yes | Positive |
| | and foam cells | | |
| 25 | Benign foam cells | Yes | Negative |
| 26 | Benign foam cells | No | Negative |
| 27 | Benign foam cells | No | Negative |
| 28 | Ductal hyperplasia | No | Negative |
| 29 | Hyperplasia with mild atypia | No | Negative |
| 30 | Benign foam cells | Yes | Negative |
| 31 | Hyperplasia and foam cells | Yes | Negative |
| 32 | Benign | No | Negative |
| 33 | Benign | No | Negative |
| 34 | Benign | No | Negative |
| 35 | Benign | No | Negative |
| 36 | Benign | No | Negative |
| 37 | Benign | Yes | Negative |
| 38 | Benign | No | Negative |
| 39 | Benign; Stromal fibrosis and duct | Yes | Negative |
| | ectasia | | |
| 40 | Benign | No | Negative |
| 41 | Benign | No | Negative |
| 42 | Benign | No | Negative |
| 43 | Duct Papilloma, duct hyperplasia | Yes | Negative |
| | without atypia | | |
| 44 | Pseudopapillary nest with moderate | Yes | Negative |
| | atypia; Hyperplasia with mild atypia | | |
| 45 | Benign | Yes | Negative |
| 46 | Benign | Yes | Negative |
| 47 | Benign | Yes | Negative |
| 48 | Benign | Yes | Negative |
| 49 | Hyperplasia with atypia | Yes | Positive |

### Example 7

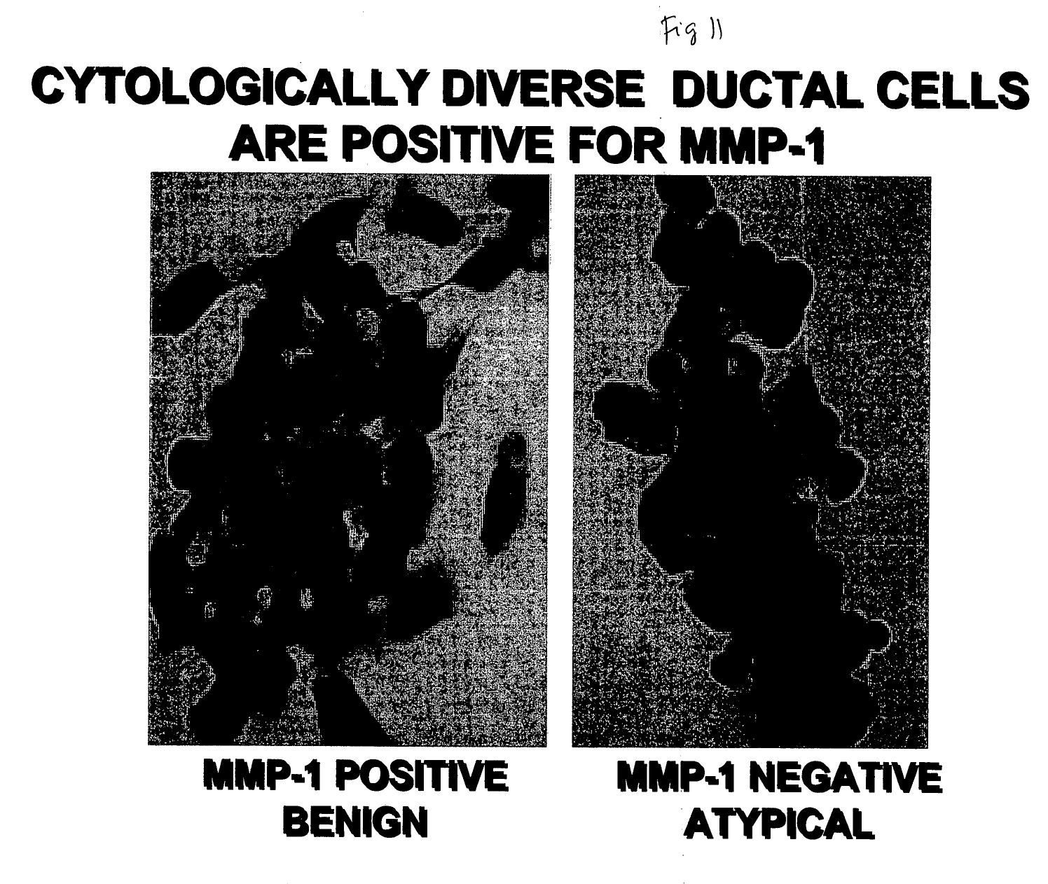

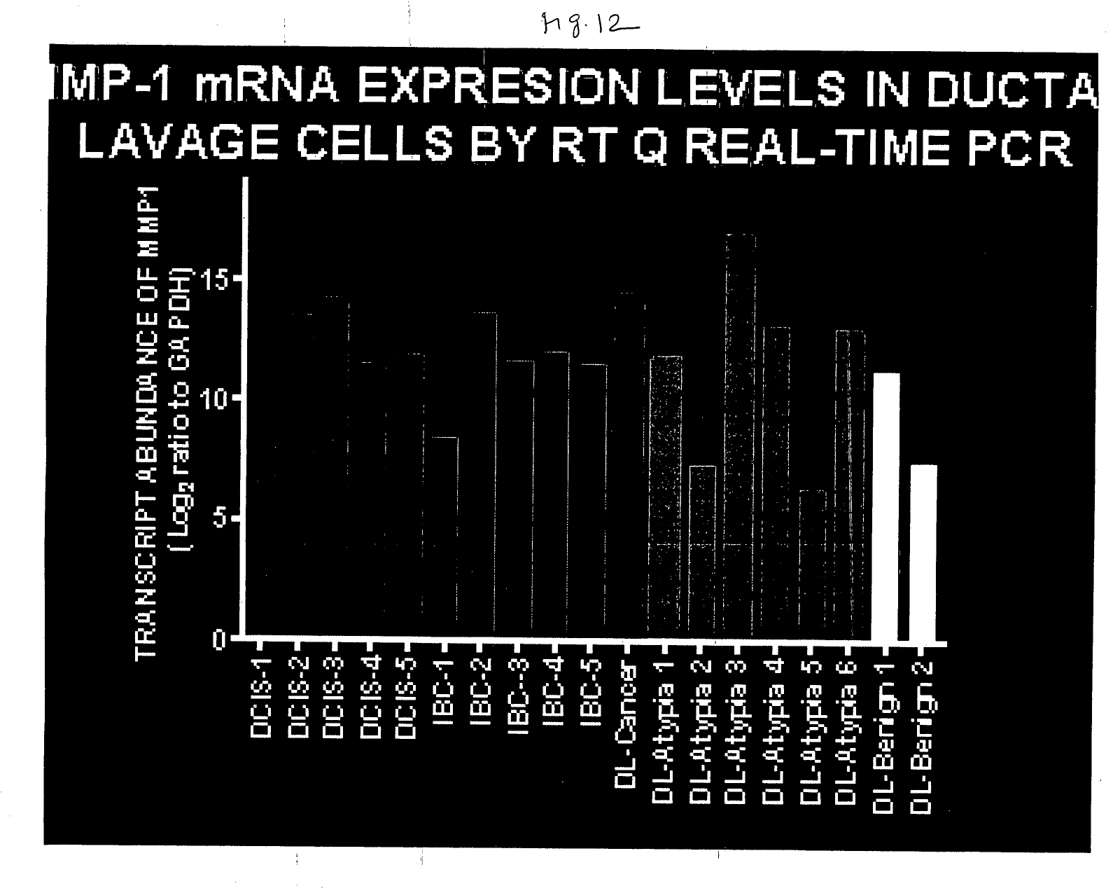

This example demonstrates the feasibility of quantifying the levels of MMP-1 mRNA in dutal cells obtained by procedures such as ductal lavage procedure. Quantification of MMP-1 mRNA in ductal lavage in comparison to the house keeping gene, Glyceroldehyde-3 Phosphate dehydrogenase (GAPDH) MRNA by quantitative real-time PCR using MMP-1 Assays-on-Demand™ Gene Expression reagents from Applied Biosystems is described. The cDNAs from six DCIS and twelve Stage I IBC samples were included as positive controls. To ensure that the amount of cDNA used in all the samples is uniform, first GAPDH transcript copy numbers were first quantified (Poola, I, Analytical Biochemistry, 314, 217-226 (2003)) and the cDNA equivalent to 5-6×10⁵ copies of GAPDH from each sample was used for quantification of MMP-1 mRNA. As expected, all DCIS, Stage I and a ductal lavage sample collected from a cancer patient were positive for this marker. In addition, ductal lavage samples from nine non-cancer subjects were also positive for MMP-1. Of the nine positive, 7 samples were diagnosed as hyperplasia to mild atypia by cytology and all of them had nipple discharge. Two samples which were diagnosed as benign were also positive for MMP-1. Three samples that were diagnosed as atypia were negative for MMP-1 (Table 6). The results of MMP-1 mRNA expression levels in ductal lavage samples by Taqman real-time PCR are shown in Table 6 and FIG. 12. These results demonstrate that the expression levels of MMP-1 mRNA levels in all the positive ductal lavage samples are comparable to the levels present in cancer tissues. These results also demonstrate the feasibility of quantifying the expression levels of MMP-1 mRNA in ductal lavage samples.

All references, including publications, patent applications, and patents, cited herein are herby incorporated by references to the same extent as if each reference were individually and specifically indicated to be incorporated by reference and were set forth in its entirely herein.

The use of the terms “a” and “an” and “the” and similar referents in the context of describing the invention (especially in the context of the following claims) are to be constructed to cover both the singular and the plural, unless otherwise indicated herein or clearly contradicted by context. The terms “comprising”, “having”, “including”, and “containing” are to be constructed as open-ended terms (i.e., meaning “including,” but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be constructed as indicating any non-claimed element as essential to the practice of the invention.

1. A method of determining a hyperproliferative condition or a predisposition to a hyperproliferative condition in a mammal, the method assessing expression of the matrix metalloproteinase 1 (MMP-1) gene product in a sample obtained from the mammal.

2. The method of claim 13, wherein expression of the MMP-1 gene product is assessed by measuring expression of MMP-1 protein.

3. (canceled)

4. The method of claim 2, wherein protein expression is measured by a method selected from western blot, functional assay, immunohistochemistry, Enzyme Linked Immunosorbant Assay (ELISA), protein profiling by 2D gel electrophoresis followed by identification by Spectroscopy, and peptide profiling after proteolysis followed by separation and identification by Spectroscopy (proteamics).

5. The method of claim 1, further comprising assessing expression of at least one other gene product selected from the group consisting of the products of the 541 genes described in FIG. 3.

6. The method of claim 1, further comprising assessing expression of at least one gene product selected from the group consisting of the Carcino Embryonic Antigen Cell Adhesion Molecule 6 (CEACAM 6) gene product, the Hyaluronoglucosaminidase I (HYAL1) gene product, and the Mitogen Activated Protein kinase kinase kinase kinase 1 (MAP4kinase 1) gene product.

7. The method of claim 13, wherein the sample comprises cells of a tissue of the mammal selected from the group consisting of breast, ovary, brain, bone, female genital tract, male genital tract, gastrointestinal tract, nervous system, and immune system tissues.

8. The method of claim 7, wherein the sample comprises breast tissue selected from the group consisting of normal tissue, benign tissue, cancer tissue, ductal hyperplastic tissue, lobular hyperplastic tissue, atypical ductal hyperplastic tissue, atypical lobular hyperplastic tissue, DCIS, and LCIS.

9. The method to of claim 7, wherein the mammal with has a condition selected from the group consisting of a history of cancer, a history of any other breast lesions, and normal breast tissue.

10. The method of claim 13, wherein the sample comprises cells obtained from ductal lavage of the breast of the mammal.

11. The method of claim 13, wherein the sample comprises cells obtained from the mammal using Random Periareolar Fine Needle Aspiration (RPFNA).

12. The method of claim 13, wherein the sample comprises cells obtained from the mammal using Core Needle Biopsy.

13. A method of screening a mammal for a predisposition to a hyperproliferative condition, the method comprising assessing expression of the matrix metalloproteinase I (MMP-1) gene product in a sample obtained from the mammal.

14. The method of claim 13, further comprising assessing expression of at least one other gene product selected from the group consisting of the products of the 541 genes described in FIG. 3.

15. The method of claim 13, further comprising assessing expression of at least one gene product selected from the group consisting of the Carcino Embryonic Antigen Cell Adhesion Molecule 6 (CEACAM 6) gene product, the hyaluronoglucosaminidase I (HYL1) gene product, and the Mitogen Activated Protein kinase kinase kinase kinase 1 (MAP4 kinase 1) gene product.

16. A method for early detection of cancer at precancerous stage in a mammal, the method comprising assessing expression of the matrix metalloproteinase 1 (MMP-1) gene product in a sample obtained from the mammal.

17-21. (canceled)

22. The method of claim 1, wherein the mammal is a human.

23. The method of claim 13, wherein the mammal is a human.

24. The method of claim 16, wherein the mammal is a human.

25. The method of claim 16, wherein the sample comprises cells of a tissue of the mammal selected from the group consisting of breast, ovary, brain, bone, female genital tract, male genital tract, gastrointestinal tract, nervous system, and immune system tissues.

26. The method of claim 25, wherein the sample comprises breast tissue selected from the group consisting of normal tissue, benign tissue, cancer tissue, ductal hyperplastic tissue, lobular hyperplastic tissue, atypical ductal hyperplastic tissue, atypical lobular hyperplastic tissue, DCIS, and LCIS.
